SEQUENCE LISTING (5 sequences)

SEQ ID NO: 1 - protein, 12 aa, Leishmania infantum
KLLVQNQGEMIK

SEQ ID NO: 2 - protein, 16 aa, Leishmania infantum
KAPSEWMGGVMGFVNK

SEQ ID NO: 3 - protein, 12 aa, Leishmania infantum
KLGQGISLIMIK

SEQ ID NO: 4 - protein, 9 aa, Leishmania infantum
KDLVPLWGR

SEQ ID NO: 5 - protein, 449 aa, Leishmania infantum
MGKDKVHMNLVVVGHVDAGKSTATGHLIYKCGGIDKRTIEKFEKEAAEIGKASFKYAWVLDKLKAERERGITIDIALWKFESPKSVFTIIDAPGHRDFIKNMITGTSQADAAILMIDSTHGGFEAGISKDGQTREHALLAFTLGVKQMVVCCNKMDDKTVTYAQSRYDEISKEVGAYLKRVGYNPEKVRFIPISGWQGDNMIERSDNMPWYKGPTLLDALDMLEPPVRPVDKPLRLPLQDVYKIGGIGTVPVGRVETGIMKPGDVVTFAPANVTTEVKSIEMHHEQLAEAQPGDNVGFNVKNVSVKDIRRGNVCGNSKNDPPKEAADFTAQVIVLNHPGQISNGYAPVLDCHTSHIACRFAEIESKIDRRSGKELEKNPKAIKSGDAAIVKMVPQKPMCVEVFNDYAPLGRFAVRDMRQTVAVGIIKGVNKKEGSGGKVTKAAAKAAKK

(19) United States
(12) Patent Application Publication
(10) Pub. No.: US 2006/0211056 A1
(43) Pub. Date: Sep. 21, 2006

(54) Leishmania antigens suitable for a diagnostic kit of Leishmania

(76) Inventors: Koussay Dellagi, La Marsa (TN); Sayda Kamoun-Essghaier, Ariana (TN); Ikram Guizani, Ariana (TN)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/337,801

(22) Filed: Jan. 24, 2006

Related U.S. Application Data
(60) Provisional application No. 60/646,496, filed on Jan. 25, 2005.

(51) Int. Cl. G01N 33/569; C07H 21/04; C12P 21/06; C12N 9/02; C12N 1/10; C07K 14/44; C07K 16/20

(52) U.S. Cl. 435/7.22; 530/350; 530/388.6; 435/69.1; 435/320.1; 435/258.1; 536/23.7; 435/189

(57) ABSTRACT

A purified Leishmania infantum polypeptide comprising at least 10 consecutive amino acids of a protein is provided. The protein is mitochondrial integral ADP/ATP carrier protein, NADH-cytochrome b5 reductase, mitochondrial carrier protein, guanine nucleotide binding protein beta subunit (LACK), aldehyde reductase, ubiquinol-cytochrome-c reductase Rieske iron-sulfur protein, truncated elongation factor 1-alpha having a molecular weight as determined by SDS-PAGE of 36.4 kDa, truncated elongation factor 1-alpha having a molecular weight as determined by SDS-PAGE of 34.5 kDa, or truncated elongation factor 1-alpha having a molecular weight as determined by SDS-PAGE of 30.6 kDa. Methods for using the polypeptides for diagnosing MVL are disclosed.

### CROSS REFERENCE TO RELATED APPLICATIONS

This application claims the benefit of U.S. Provisional Application No. 60/646,496, filed Jan. 25, 2005, the content of which is incorporated herein by reference.

### DESCRIPTION OF THE INVENTION

1. Field of the Invention

This invention is directed to purified and isolated polypeptides, antibodies generated against these polypeptides, diagnostic kits and methods for detecting the presence or absence of antibodies, which bind to these polypeptides, immunogenic compositions comprising these polypeptides, mixtures of polypeptides, methods of diagnosis, and methods of identifying polypeptides.

2. Background of the Invention

Trypanosomatid protozoans belonging to the genus Leishmania are obligate parasites of mammalian macrophages. The life cycle of these organisms go through two morphologically different stages: the amastigote, found in the parasitophorous vacuoles of host macrophages and dentritic cells, and the promastigote, which is an extracellular flagellated form, found in the gut of the sandfly vector. At least 15 Leishmania species are infectious to humans and cause a large spectrum of diseases, including cutaneous, mucocutaneous, and visceral leishmaniasis, as well as asymptomatic infections. Intermediate forms may be encountered, and the same parasite species may cause different forms of disease. Leishmaniases are prevalent in four continents and are considered by the World Health Organisation among the major infectious diseases. In 1990, the World Health Organisation estimated that ˜350 million people were at risk of acquiring leishmaniasis and that 12 million were currently infected (1).

In Tunisia, as in other Mediterranean countries, several forms of leishmaniasis coexist among which is Mediterranean visceral leishmaniasis (MVL) caused by Leishmania infantum. MVL, also known as infantile kala-azar, is a severe systemic disease, which affects mostly children under the age of five and is constantly fatal if not rapidly diagnosed and treated. MVL is usually featured clinically as a combination of prolonged fever, hepato-splenomegaly, anaemia, and leucopoenia (5). It is characterized by high titers of both non-specific and specific antibodies (10). Early diagnosis is of great importance for effective treatment of this potentially fatal disease. The diagnosis of MVL is based on the demonstration of amastigotes in Giemsa stained smears of bone marrow aspirates or needle punctions of the spleen (12, 44), and by growing parasites on Novy, McNeal et Nicolle (NNN) medium. The major drawback of these two classical diagnostic tests are their weak sensitivities.

Several serological tests are of diagnostic value, including the indirect immunofluorescence assay (4), the direct agglutination test (19), and the enzyme-linked immunosorbent assays (ELISA) using whole parasites or crude antigens (3, 13) or immunoblot analysis (13, 22, 53, 60). These methods proved to be more sensitive than the existing invasive techniques for MVL diagnosis. One drawback of serological assays using whole parasites relates to the existence of cross-reactivity with other pathogens including Trypanosoma cruzi, Mycobacteria, malaria parasites, or amoeba, which are coendemic to Leishmania in many parts of the world (7, 51). Performance of serodiagnosis could be improved using purified or recombinant leishmanial antigens such as: gp63 (40, 56), Hsp70 (30, 48), p94 (53), gp70 and p72 (24), p32 (61), rK39 (2, 11) r gene B protein (rGBP) (15, 31), H2A and H2B (31, 57, 58, 59), rLACK (31), and the promastigote surface antigen-2 (rPSA-2) (31), or synthetic peptides (14, 48) and antigens from promastigotes conditioned media (33).

A 32 kDa fraction (P32) of Leishmania infantum promastigotes membranes consistently reacts on Western blots with sera from MVL patients, but not with sera from patients with zoonotic cutaneous leishmaniasis (ZCL) (61). Interestingly, P32 kDa antigen(s) did not react with sera from patients suffering from other infectious diseases like toxoplasmosis, echinococcosis, or tuberculosis. When electroeluted and tested on ELISA, the P32 kDa band had good performances in terms of specificity and sensitivity (94% each) and showed some cross-reactivity only with sera from patients with Chagas disease. Only 1.4% false positives were observed with P32 as compared to 19% or 7.3% when using crude membrane or soluble antigens respectively. Moreover, the antibody response to P32 antigen appeared to be specific to patients having an overt disease as compared to asymptomatic subjects.

For these reasons, there is a considerable need for the development of specific, sensitive, and rapid diagnosis of MVL patients. Additionally, there is a need for techniques to identify useful Leishmania polypeptides for use in serological assays. Such an understanding may also lead to effective means for treating or controlling Leishmania infection.

The following abbreviations are used herein: IEF: isoelectrofocalisation, IMAs: integral membrane antigens, LC-MS/MS: Liqui chromatography Mass Spectrometry, MVL: Mediterranean visceral leishmaniasis, MBAs: Membrane antigens, NEPHGE: non equilibrium pH gradient electrophoresis, PMAs: peripheral membrane antigens, PVDF: polyvinylidene difluoride, SAs: soluble antigens, TAs: total antigens, 2DE: two-dimensional gel electrophoresis, ZCL: Zoonotic cutaneous leishmaniasis.

### SUMMARY OF THE INVENTION

Accordingly, this invention aids in fulfilling these needs in the art. The invention encompasses a purified Leishmania infantum polypeptide comprising at least 10 consecutive amino acids of a protein, wherein the protein is mitochondrial integral ADP/ATP carrier protein, NADH-cytochrome b5 reductase, mitochondrial carrier protein, guanine nucleotide binding protein beta subunit (LACK), aldehyde reductase, ubiquinol-cytochrome-c reductase Rieske iron-sulfur protein, truncated elongation factor 1-alpha having a molecular weight as determined by SDS-PAGE of 36.4 kDa, truncated elongation factor 1-alpha having a molecular weight as determined by SDS-PAGE of 34.5 kDa, or truncated elongation factor 1-alpha having a molecular weight as determined by SDS-PAGE of 30.6 kDa.

The invention also encompasses a polypeptide, wherein the polypeptide comprises at least 20 consecutive amino acids, or at least 30 consecutive amino acids, of a protein of the invention. The invention includes a polypeptide comprising a Leishmania immunodominant antigen, and also includes recombinant polypeptides.

The invention further encompasses a purified antibody that binds to at least one polypeptide of the invention. This antibody can be a monoclonal antibody, or a polyclonal antibody.

The invention also provides for a method for diagnosing Mediterranean visceral leishmaniasis (MVL), wherein the method comprises providing a composition comprising biological material suspected of being infected with Leishmania infantum, and assaying for the presence of antigens in the biological material that are immunologically reactive with an antibody of the invention.

The invention also encompasses a diagnostic kit for detecting the presence or absence of antibodies which bind to Leishmania comprising at least one polypeptide of the invention, and means for detecting the formation of an immune complex between the polypeptide and antibodies, wherein the means are present in an amount sufficient to perform said detection. A suitable detection means includes, for example, an indirect immunofluorescence assay, a direct agglutination test, or an enzyme-linked immunosorbent assay (ELISA).

The invention also encompasses an immunogenic composition comprising at least one polypeptide of the invention in an amount sufficient to induce an immunogenic or protective response in vivo, and a pharmaceutically acceptable carrier therefor. This composition can comprise a neutralizing amount of the polypeptide.

The invention also encompasses an immunological complex comprising a polypeptide of the invention, and an antibody that specifically recognizes the polypeptide.

The invention also provides for a method for diagnosing Mediterranean visceral leishmaniasis (MVL), wherein the method comprises providing a composition comprising biological material suspected of being infected with Leishmania infantum, and assaying for the presence of antibodies in the biological material that are immunologically reactive with a polypeptide of the invention. The invention further encompasses an in vitro diagnostic method for the detection of the presence or absence of antibodies, which bind to a polypeptide of the invention, wherein the method comprises contacting the polypeptide with a biological fluid for a time and under conditions sufficient for the polypeptide and antibodies in the biological fluid to form an antigen-antibody complex, and detecting the formation of the complex. This in vitro diagnostic method can involve measuring the formation of the antigen-antibody complex, for example, by immunoassay based on Western blot technique, ELISA, indirect immunofluoresence assay, or immunoprecipitation assay.

The invention also encompasses a mixture of purified Leishmania infantum polypeptides comprising at least two of mitochondrial integral ADP/ATP carrier protein, NADH-cytochrome b5 reductase, mitochondrial carrier protein, guanine nucleotide binding protein beta subunit (LACK), aldehyde reductase, ubiquinol-cytochrome-c reductase Rieske iron-sulfur protein, truncated elongation factor 1-alpha having a molecular weight as determined by SDS-PAGE of 36.4 kDa, truncated elongation factor 1-alpha having a molecular weight as determined by SDS-P AGE of 34.5 kDa, or truncated elongation factor 1-alpha having a molecular weight as determined by SDS-PAGE of 30.6 kDa, wherein the mixture is essentially free of lipids. In one embodiment, each of the polypeptides in the mixture has a molecular weight of approximately 30-36 kDa as determined by SDS-PAGE. In another embodiment, the polypeptides comprise Leishmania immunodominant antigens.

The invention further encompasses the mixture, wherein the polypeptides are recombinant polypeptides.

The invention also encompasses a diagnostic kit for Mediterranean visceral leishmaniasis (MVL), wherein the kit comprises the mixture, and means for detecting the formation of immune complex between antigen and antibodies, wherein the means are present in an amount sufficient to perform said detection. The means for detecting formation of immune complex between the antigen and antibodies can comprise, for example, an indirect immunofluorescence assay, a direct agglutination test, or an enzyme-linked immunosorbent assay (ELISA).

The invention also provides for a method for identifying a polypepitde from Leishmania promastigotes comprising the steps of:

a) providing Leishmania membrane antigens;

b) electrophoresing the membrane antigens;

c) transferring the membrane antigens to a suitable surface;

e) contacting the membrane antigens with Mediterranean visceral leishmaniasis (MVL) sera and Zoonotic cutaneous leishmaniasis (ZVL) sera under conditions sufficient to form antigen-antibody complexes;

f) detecting the formation of the antigen-antibody complexes; and

g) identifying a polypeptide that reacts with MVL sera, but not ZCL sera.

In one embodiment, identifying a polypeptide that reacts with MVL sera but not ZCL sera comprises amino acid sequencing. In another embodiment, it comprises liquid chromatography mass spectrometry. In another embodiment, the polypeptide forms a complex with antibodies in the biological fluid from Mediterranean visceral leishmaniasis patients, and does not form a complex with antibodies in the biological fluid from patients infected with Trypanosoma cruzi, Mycobacteria, malaria parasites, or amoeba. In yet another embodiment, the electrophoresis comprises two dimensional non equilibrium pH gradient electrophoresis.

The method can further comprise detecting a polypeptide-antibody complex by immunoassay based on Western blot technique, ELISA, indirect immunofluorescence assay, or immunoprecipitation assay.

The invention also encompasses a purified nucleic acid encoding a polypeptide comprising at least 10 consecutive amino acids of a protein, wherein said protein is mitochondrial integral ADP/ATP carrier protein, NADH-cytochrome b5 reductase, mitochondrial carrier protein, guanine nucleotide binding protein beta subunit (LACK), aldehyde reductase, ubiquinol-cytochrome-c reductase Rieske iron-sulfur protein, truncated elongation factor 1-alpha having a molecular weight as determined by SDS-PAGE of 36.4 kDa, truncated elongation factor 1-alpha having a molecular weight as determined by SDS-PAGE of 34.5 kDa, or truncated elongation factor 1-alpha having a molecular weight as determined by SDS-PAGE of 30.6 kDa.

Additionally, the invention encompasses a purified nucleic acid molecule that hybridizes to either strand of this nucleic acid under conditions of moderate stringency in 50% formamide and 6×SSC at 42° C., with washing conditions of 0.5×SSC and 0.1% SDS at 60° C., as well as a purified nucleic acid molecule that hybridizes to either strand of this nucleic acid under conditions of high stringency in 50% formamide and 6×SSC at 42° C., with washing conditions of 0.2×SSC and 0.1% SDS at 68° C.

The invention further provides for a purified nucleic acid molecule, which encodes mitochondrial integral ADP/ATP carrier protein, NADH-cytochrome b5 reductase, mitochondrial carrier protein, guanine nucleotide binding protein beta subunit (LACK), aldehyde reductase, ubiquinol-cytochrome-c reductase Rieske iron-sulfur protein, truncated elongation factor 1-alpha having a molecular weight as determined by SDS-PAGE of 36.4 kDa, truncated elongation factor 1-alpha having a molecular weight as determined by SDS-PAGE of 34.5 kDa, or truncated elongation factor 1-alpha having a molecular weight as determined by SDS-PAGE of 30.6 kDa.

The invention also provides for recombinant vectors that direct the expression of these nucleic acids.

It is to be understood that both the foregoing general description and the following detailed description are exemplary and explanatory only and are not restrictive of the invention, as claimed.

### BRIEF DESCRIPTION OF THE DRAWINGS

This invention will be described in detail with reference to the drawings in which:

FIG. 1 shows a Western blot analysis of MBAs extracted from L. infantum promastigotes, revealed by MVL patient sera pool (a) or ZCL sera pool (b). Molecular weights are indicated in kDa. The frame indicates the antigenic fraction P30-P36 of interest in this invention.

FIG. 2 shows Western blot analyses of the P30-P36 solubilized fraction from membrane antigens. Panel A illustrates the different steps of pH 11-treatment of the membrane antigens (MBAs), which correspond to the pellet after centrifugation of total antigens at 10,000 g for 30 min. The MBAs were treated at pH 11 and centrifuged at 90,000 g for 3 h at +4° C. Integral membranes (P_{90,000} fraction) and peripheral membranes (S_{90,000} fraction) correspond to (IMAs) and (PMAs), respectively. The pH 11 treatment partially solubilizes antigen(s) at 36 kDa. Panel B illustrates the protein content of supernatants obtained after treatment of IMAs by six different detergents (CHAPS, TX-100, DIG, OG, Na Cholate or LDAO) and centrifugation at 90,000 g for 1 h 30 min at +4° C. Each fraction was analysed by immunoblotting using the MVL sera pool. Only LDAO partially solubilizes the antigens at 32 kDa.

FIG. 3 shows a Western blot analysis of promastigote membrane antigens, prepared from L. infantum, separated by SDS-PAGE, and incubated with anti-sera raised in rabbits against P1 (KLLVQNQGEMIK [SEQ ID NO: 1]), P2 (KAPSEWMGGVM/GFVNK [SEQ ID NO: 2]), P3 (KLGQGISLIMIK [SEQ ID NO: 3]) and P4 (KDLVPLWGR [SEQ ID NO: 4]) peptide (lane 2, 4, 6, 8). Pre-immune rabbit sera were used as controls (lanes 1, 3, 5 and 7). Protein molecular mass markers are shown on the left. The anti-P1, P2 and P3 sera react with Leishmania products. The anti-P4 serum does not react with Leishmania membrane antigens.

FIG. 4 is a comparative analysis of the reactivity of MVL sera pool and rabbit antipeptide sera, diluted at 1/2000 and 1/20000 respectively, to L. infantum promastigote membrane antigen extracts. Membrane antigen extracts (400 μg) were separated in the first dimension by NEPHGE/2D-PAGE using ampholyte ratio (1-3) for pH 3:10 and 6/8, respectively. Transferred polypeptides were first incubated with MVL sera pool and revealed (Blot A). After stripping, it was further incubated with anti P3 rabbit serum (panel D). A second blot was first incubated with anti-P1 (panel B), then after stripping, incubated with anti-P2 (panel C).

FIG. 5 shows a 2-D analysis of Commassie blue stained membrane antigens from L. infantum promastigotes (A) and Western blot of the same antigens incubated with the MVL sera pool diluted at 1/2000 (B). The membrane proteins (400 μg) were separated by NEPHGE in the first dimension and by SDS-PAGE in the second one. The arrows indicate the immunoreactive membrane proteins, which are visible in Coomassie stained gels in the 30-36 kDa area (spots a-f) and at 49 kDa (spots g and h₁-h₃). The apparent molecular mass of marker proteins and the non equilibrium pH range at the end of migration are indicated on the left and above the panels, respectively.

FIG. 6 is an alignment of the matching peptides to the sequence of L. donovani elongation factor 1-alpha promastigotes (AN: Q95VF2). Sequences underlined indicate the matching peptides identified for spots a, b and c. Sequences in bold indicate the matching peptides for spots g, h₁, h₂ and h₃.

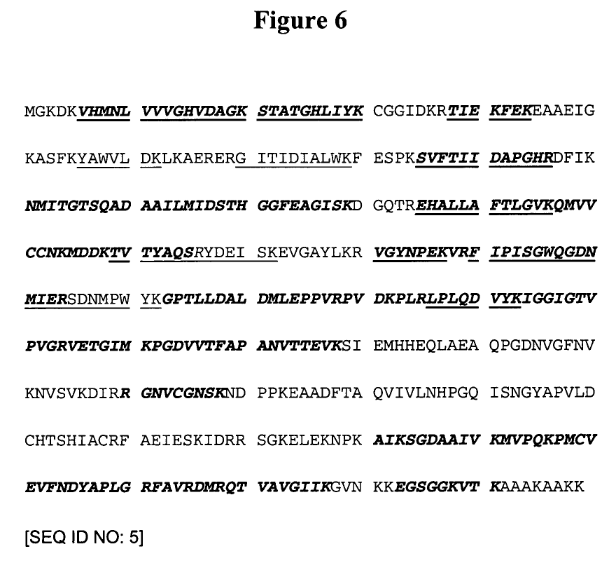

### DETAILED DESCRIPTION OF THE INVENTION

Identified and characterized were polypeptides of a 30-36 kDa fraction of L. infantum promastigote membranes known to be immunodominant in Mediterranean Visceral leishmaniasis (MVL). These polyptides can serve as consistent and reliable serological markers of this disease.

In a first approach, Coomassie-stained protein bands (P32 and P33 kDa), which specifically react by immunoblot with sera from MVL patients, were excised from the gel and submitted to enzymatic digestion to generate peptides. Four peptides were sequenced, three of which were shown to be definitely associated to MVL reactive antigens, and ascribed to a mitochondrial integral ADP/ATP carrier protein from L. major, a putative NADH-cytochrome b5 reductase and a putative mitochondrial carrier protein.

A second approach combined two-dimensional gel electrophoresis (2DE) of membrane antigens and mass spectrometry (LC-MS/MS) using a quadruple time-of-flight (Q-TOF) analysis. Six immunoreactive spots, resolved within the 30-36 kDa molecular mass and 6.7-7.4 pH ranges, corresponded to four Leishmania products. Sequences derived from two spots were ascribed to guanine nucleotide binding protein beta subunit-like known as the activated protein kinase C receptor homolog antigen LACK and to a probable member of the aldehyde reductase family. One spot was identified as a probable ubiquinol-cytochrome-c reductase (EC 1.10.2.2) Rieske iron-sulfur protein precursor. The remaining three spots were identified as truncated forms of elongation factor-1α.

Methods for the detection of anti-leishmanial antibodies in sera are generally considered to provide much better sensitivities (80% or greater) than the two methods considered as the gold standards for the diagnosis of VL, namely microscopy and culture. However, serological assays, based on the use of whole organism or crude leishmanial or semi-purified leishmanial antigens are associated to false positive due to cross-reactivity with other microorganisms (26). Furthermore, these antigens are not well defined; thus, variation in purification conditions might qualitatively influence the performance of the test particularly through parameters like stability, sensitivity and reproducibility. Therefore, there is a need to use well-defined and reliable diagnostic tests based on Leishmania specific peptides or antigens (2, 11, 14, 48, 56). The new generation of diagnostic tests should also be affordable and easy to implement in remote, poor and less developed rural endemic areas.

The use of patient's sera with screening systems like Western blots or expression libraries has allowed selection of semi-purified or recombinant antigens (13, 22, 54, 56, 61). This invention targets the semi-purified p32 fraction known to have good statistical performances in ELISA assays (61). Considering that this fraction may actually contain several antigens, this invention characterized its components using two proteomic approaches.

Each one of the two approaches developed presented advantages and drawbacks. The micro-sequencing of peptides deriving from the digestion of the immunodominant bands purified from 1D electrophoresis appeared as a lengthy procedure. It allowed indirect identification of the antigens on the basis of a search for identities of a short peptide sequence against databases; the significance of the hits observed being defined by its relative score and e-value. A priori, the relationship between the different peptides sequenced for a given band was difficult to establish as the bands had heterogeneous polypeptide content. For the same reason, it was also difficult to ascertain that the peptides identified were immunogenic and contributed to eliciting the polyclonal humoral immune response during the disease. These properties were assessed upon the chemical synthesis of the peptides, its coupling, the generation of rabbit polyclonal sera, and the comparison of the antipeptide and MVL sera reactivities on immunoblots of MBAs separated by 1D or 2D electrophoresis. Only 3 out of the 4 peptides sequenced were shown to bear antigenic properties and to correspond to targets of the humoral immune response of MVL patients.

The second approach was based on two-dimensional (NEPHGE/SDS-PAGE) separation of the antigenic fraction and the subsequent LC-MS/MS analysis of immunodominant spots purified from Coomassie-stained gels. The ten spots analysed by this way allowed the unambiguous identification of 4 Leishmania products and confirmed the power of this approach. First, the 2D separation increased the resolution of the polypeptidic content of the bands within the 5.85-7.6 pH and 30-36 kDa ranges, making it possible to differentiate at least 14 immunogenic polypeptide spots. Second, the abundant antigens, detected on and picked from Coomassie-stained gels were analysed by LC-MS/MS, a sensitive and powerful method that uses mass spectra to assign the polypeptidic content of each spot to proteins deposited in data banks. The significance of the hits was evaluated by various statistical parameters like the Mowse score, the percentage of sequence coverage, which allowed unambiguous identification. The Mowse scores of the first hit for each polypeptide was so significant and so divergent from the range of scores of the following hits that one could confidently consider the first hit as significant. Furthermore, several peptides generated from the various spots usually 4 to 19, of 6 to 29 residues length matched these hits resulting in a significant range of sequence coverage (8 to 53%). Indeed, this last parameter is influenced by any kind of modification occurring on any of the residues of the peptides. Modifications alter the mass of the peptide therefore deviating the relative scores which allow identification of the peptides and consequently its matching. Furthermore, the approach is powerful enough to detect overlapping polypeptides whenever this occurs. Two such spots were observed (d and f) that redundantly and concomitantly identified two known antigens, up-regulated products in the promastigote stage, the LACK antigen and a member of the aldehyde reductase family, (6, 25, 55).

Except for elongation factor 1-alpha, all identified antigens had an expected molecular mass that fitted within the range of 30-36 kDa. Furthermore, different spots were shown to correspond to the same antigen, an indication for the likely presence of isoforms within the preparation. For elongation factor 1-alpha, there was a discrepancy between the expected MM (49 kDa) and the actual one observed on gels at 32 kDa. The presence, in the preparation, of elongation factor 1-alpha at the expected size was confirmed by analysing four 49-50 kDa spots (g, h₁, h₂ and h₃), which strongly reacted with the MVL sera. The matching peptides of the spots a, b and c could be aligned only to the N-terminal part of the sequence of the EF-1α protein, which argued in favor of the existence of truncated forms of the EF-1α protein in the promastigote membrane preparations. The consultation of the L. major specific gene data base (gene DB) actually identified genes coding for truncated, N-terminal forms of the elongation factor 1-alpha, which confirmed the hypothesis. It is noteworthy that EF-1α of L. donovani was recently described as a Leishmania virulence factor. This factor is able to activate tyrosine phosphatase-1 (SHP-1), containing the Src homology 2 domain, leading to deactivation of the infected macrophages. The 49 kDa EF-1α Leishmania protein was shown to diffuse into the cytosol of infected macrophages where it exerts this activity (38). No information has been provided as to whether the naturally truncated EF-1α products could also act as virulence factors and whether they could be expressed in a stage specific manner. However, this invention shows that they are at least present at the late growth phase of the promastigote stage.

The antigens identified in the present invention, correspond to evolutionary conserved proteins encountered in cells as part of multicomponent complexes that could be considered as panantigens (a term coined earlier by Requana and co-authors (52)). This invention identified antigens that were previously known to be integral components of the mitochondrial membranes, such as mitochondrial carrier proteins, NADH-cytochrome b5 reductase and ubiquinol-cytochrome-c reductase (EC 1.10.2.2) Reiske iron-sulfur precursor. The other proteins are tightly associated to multicomponent complexes. Indeed, Gonzalez-Aseguinolaza et al. (17) showed that the LACK p36, although structurally not membrane associated, is organized within large complexes, which co-purify with membranes. This is not discordant with the results of this invention, as treatment of the membrane preparation at pH 11 liberated only partially a p36 product.

The EF-1α is actually described as being a highly abundant protein in eukaryotic cells, having a 49-50 kDa (greater than 0.4% of total protein) (27), which interacts with the cytoskeleton by binding and bundling actin filaments and microtubules, and which is associated to the endoplasmic reticulum membrane by phosphatidylinositol (20, 27, 63). Therefore, if such interactions hold true for Leishmania parasites, it would account for the association of the different forms of EF-1 alpha with our membrane preparations.

The description of elongation factor 1-alpha, its truncated N terminal parts and mitochondrial proteins as potent antibody inducers during visceral leishmaniasis corresponds to a novel finding. Interestingly, a homologous protein to EF1-alpha was found suitable for the antibody diagnosis of hymenopteran parasitism (60). Other elongation factors like EF2 or EF1 beta/delta were also shown to be associated with elicitation of cellular immune responses in leishmaniasis patients (47) or allergic manifestations in patients having cystic echinococcosis due to Echinococcus granulosus (32, 42), respectively.

As used herein, the term polypeptides refers to a genus of polypeptides that encompasses proteins having the amino acid sequences of Leishmania infantum mitochondrial integral ADP/ATP carrier protein, NADH-cytochrome b5 reductase, mitochondrial carrier protein, guanine nucleotide binding protein beta subunit (LACK), aldehyde reductase, ubiquinol-cytochrome-c reductase Rieske iron-sulfur protein, truncated elongation factor 1-alpha having a molecular weight as determined by SDS-PAGE of 36.4 kDa, truncated elongation factor 1-alpha having a molecular weight as determined by SDS-PAGE of 34.5 kDa, or truncated elongation factor 1-alpha having a molecular weight as determined by SDS-PAGE of 30.6 kDa. The term polypeptides further encompasses all amino acid fragments derived from any of these amino acid sequences.

The term “purified” as used herein, means that the polypeptides of the invention are essentially free of association with other proteins or polypeptides, for example, as a purification product of recombinant host cell culture or as a purified product from a non-recombinant source.

The term “substantially purified” as used herein, refers to a mixture that contains the polypeptides of the invention and is essentially free of association with other proteins or polypeptides, but for the presence of known proteins that can be removed using a specific antibody, and which substantially purified polypeptides can be used as antigens.

The term “essentially free of lipids” as used herein, refers to a mixture that contains the polypeptides of the invention and is essentially free of association with lipids.

A polypeptide “variant” as referred to herein means a polypeptide substantially homologous to any one of the native polypeptides of the invention, but which have an amino acid sequence different from these polypeptides because of one or more deletions, insertions, or substitutions. The variant amino acid sequence preferably is at least 95% identical to one of the polypeptides amino acid sequence, most preferably at least 98% identical. The percent identity can be determined, for example by comparing sequence information using the GAP computer program, version 6.0 described by Devereux et al. (Nucl. Acids Res. 12:387, 1984) and available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math 2:482, 1981). The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp. 353-358, 1979; (2) a penalty of 3.0 for each gap and an additional 0.10 penalty for each symbol in each gap; and (3) no penalty for end gaps.

Variants can comprise conservatively substituted sequences, meaning that a given amino acid residue is replaced by a residue having similar physiochemical characteristics. Examples of conservative substitutions include substitution of one aliphatic residue for another, such as lie, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring variants of the polypeptides of the invention are also encompassed by the invention. Examples of such variants are proteins that result from alternate mRNA splicing events or from proteolytic cleavage of the polypeptides of the invention. Variations attributable to proteolysis include, for example, differences in the termini upon expression in different types of host cells, due to proteolytic removal of one or more terminal amino acids from the polypeptides of the invention. Variations attributable to frameshifting include, for example, differences in the termini upon expression in different types of host cells.

As stated above, the invention provides isolated and purified, or homogeneous, polypeptides, both recombinant and non-recombinant. Variants and derivatives of native polypeptides that can be used as antigens can be obtained by mutations of nucleotide sequences coding for the native polypeptides of the invention. Alterations of the native amino acid sequence can be accomplished by any of a number of conventional methods. Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site-specific mutagenesis procedures can be employed to provide an altered gene wherein predetermined codons can be altered by substitution, deletion, or insertion. Exemplary methods of making the alterations set forth above are disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); Kunkel (Proc. Natl. Acad. Sci. USA 82:488, 1985); Kunkel et al. (Methods in Enzymol. 154:367, 1987); and U.S. Pat. Nos. 4,518,584 and 4,737,462, all of which are incorporated by reference.

Within an aspect of the invention, the polynucleotides or polypeptides of the invention can be utilized to prepare antibodies that specifically bind to the polypeptides of the invention. The term “antibodies” is meant to include polyclonal antibodies, monoclonal antibodies, fragments thereof such as F(ab′)2 and Fab fragments, as well as any recombinantly produced binding partners. Antibodies are defined to be specifically binding if they bind any one of the polypeptides with a Kₐ of greater than or equal to about 10⁷ M⁻¹. Affinities of binding partners or antibodies can be readily determined using conventional techniques, for example, those described by Scatchard et al., Ann. N.Y Acad. Sci., 51:660 (1949). Polyclonal antibodies can be readily generated from a variety of sources, for example, horses, cows, goats, sheep, dogs, chickens, rabbits, mice, or rats, using procedures that are well known in the art.

It will be understood that the present invention is intended to encompass the previously described proteins in isolated or purified form, whether obtained using the techniques described herein or other methods. In a preferred embodiment of this invention, the polypeptides are substantially free of human tissue and human tissue components, nucleic acids, extraneous proteins and lipids, and adventitious microorganisms, such as bacteria and viruses. It will also be understood that the invention encompasses equivalent proteins having substantially the same biological and immunogenic properties. Thus, this invention is intended to cover serotypic variants of the proteins of the invention.

Depending on the use to be made of the polypeptides of the invention, it may be desirable to label them. Examples of suitable labels are radioactive labels, enzymatic labels, fluorescent labels, chemiluminescent labels, and chromophores. The methods for labeling proteins and glycoproteins of the invention do not differ in essence from those widely used for labeling immunoglobulin. The need to label may be avoided by using labeled antibody to the antigen of the invention or anti-immunoglobulin to the antibodies to the antigen as an indirect marker.

Once the polypeptides of the invention have been obtained, they can be used to produce polyclonal and monoclonal antibodies reactive therewith. Thus, a protein or polypeptide of the invention can be used to immunize an animal host by techniques known in the art. Such techniques usually involve inoculation, but they may involve other modes of administration. A sufficient amount of the protein or the polypeptide is administered to create an immunogenic response in the animal host. Any host that produces antibodies to the antigen of the invention can be used. Once the animal has been immunized and sufficient time has passed for it to begin producing antibodies to the antigen, polyclonal antibodies can be recovered. The general method comprises removing blood from the animal and separating the serum from the blood. The serum, which contains antibodies to the antigen, can be used as an antiserum to the antigen. Alternatively, the antibodies can be recovered from the serum. Affinity purification is a preferred technique for recovering purified polyclonal antibodies to the antigen, from the serum.

Monoclonal antibodies to the antigens of the invention can also be prepared. One method for producing monoclonal antibodies reactive with the antigens comprises the steps of immunizing a host with the antigen; recovering antibody producing cells from the spleen of the host; fusing the antibody producing cells with myeloma cells deficient in the enzyme hypoxanthine-guanine phosphoribosyl transferase to form hybridomas; select at least one of the hybridomas by growth in a medium comprising hypoxanthine, aminopterin, and thymidine; identifying at least one of the hybridomas that produces an antibody to the antigen, culturing the identified hybridoma to produce antibody in a recoverable quantity; and recovering the antibodies produced by the cultured hybridoma.

These polyclonal or monoclonal antibodies can be used in a variety of applications. Among these is the neutralization of corresponding proteins. They can also be used to detect antigens in biological preparations or in purifying corresponding proteins, glycoproteins, or mixtures thereof, for example when used in affinity chromatographic columns.

The polypeptides of the invention can be used as antigens to identify antibodies to Leishmania in materials and to determine the concentration of the antibodies in those materials. Thus, the antigens can be used for qualitative or quantitative determination of Leishmania in a material. Such materials of course include human tissue and human cells, as well as biological fluids, such as human body fluids, including human sera. When used as a reagent in an immunoassay for determining the presence or concentration of the antibodies to Leishmania, the antigens of the present invention provide an assay that is convenient, rapid, sensitive, and specific.

More particularly, the antigens of the invention can be employed for the detection of Leishmania by means of immunoassays that are well known for use in detecting or quantifying humoral components in fluids. Thus, antigen-antibody interactions can be directly observed or determined by secondary reactions, such as precipitation or agglutination. In addition, immuno-electrophoresis techniques can also be employed. For example, the classic combination of electrophoresis in agar followed by reaction with anti-serum can be utilized, as well as two-dimensional electrophoresis, rocket electrophoresis, and immunolabeling of polyacrylamide gel patterns (Western Blot or immunoblot). Other immunoassays in which the antigens of the present invention can be employed include, but are not limited to, radioimmunoassay, competitive immunoprecipitation assay, enzyme immunoassay, and immunofluorescence assay. It will be understood that turbidimetric, colorimetric, and nephelometric techniques can be employed.

Immunoassays can be carried out by immobilizing one of the immunoreagents, either an antigen of the invention or an antibody of the invention to the antigen, on a carrier surface while retaining immunoreactivity of the reagent. The reciprocal immunoreagent can be unlabeled or labeled in such a manner that immunoreactivity is also retained. These techniques are especially suitable for use in enzyme immunoassays, such as enzyme linked immunosorbent assay (ELISA) and competitive inhibition enzyme immunoassay (CIEIA).

When either the antigen of the invention or antibody to the antigen is attached to a solid support, the support is usually a glass or plastic material. Plastic materials molded in the form of plates, tubes, beads, or disks are preferred. Examples of suitable plastic materials are polystyrene and polyvinyl chloride. If the immunoreagent does not readily bind to the solid support, a carrier material can be interposed between the reagent and the support. Examples of suitable carrier materials are proteins, such as bovine serum albumin, or chemical reagents, such as gluteraldehyde or urea. Coating of the solid phase can be carried out using conventional techniques.

The invention provides immunogenic polypeptides, and more particularly, protective polypeptides for use in the preparation of vaccine compositions against Leishmania. These polypeptides can thus be employed as vaccines by administering the polypeptides to a mammal susceptible to Leishmania infection. Conventional modes of administration can be employed. For example, administration can be carried out by oral, respiratory, or parenteral routes. Intradermal, subcutaneous, and intramuscular routes of administration are preferred when the vaccine is administered parenterally.

The ability of the polypeptides and vaccines of the invention to induce protective levels of neutralizing antibody in a host can be enhanced by emulsification with an adjuvant, incorporating in a liposome, coupling to a suitable carrier, or by combinations of these techniques. For example, the polypeptides of the invention can be administered with a conventional adjuvant, such as aluminum phosphate and aluminum hydroxide gel, in an amount sufficient to potentiate humoral or cell-mediated immune response in the host. Similarly, the polypeptides of the invention can be bound to lipid membranes or incorporated in lipid membranes to form liposomes. The use of nonpyrogenic lipids free of nucleic acids and other extraneous matter can be employed for this purpose.

The immunization schedule will depend upon several factors, such as the susceptibility of the host to infection and the age of the host. A single does of the vaccine of the invention can be administered to the host or a primary course of immunization can be followed in which several doses at intervals of time are administered. Subsequent doses used as boosters can be administered as need following the primary course.

The polypeptides and vaccines of the invention can be administered to the host in an amount sufficient to prevent or inhibit Leishmania infection or replication in vivo. In any event, the amount administered should be at least sufficient to protect the host against substantial immunosuppression, even though Leishmania infection may not be entirely prevented. An immunogenic response can be obtained by administering the proteins or glycoproteins of the invention to the host in an amount of about 10 to about 500 micrograms antigen per kilogram of body weight, preferably about 50 to about 100 micrograms antigen per kilogram of body weight. The proteins and vaccines of the invention can be administered together with a physiologically acceptable carrier. For example, a diluent, such as water or a saline solution, can be employed.

Another aspect of the invention provides a method of DNA vaccination. The method also includes administering any combination of nucleic acids encoding the a polypeptide of the invention, the proteins and polypeptides per se, with or without carrier molecules, to an individual. In embodiments, the individual is an animal, and is preferably a mammal. More preferably, the mammal is selected from the group consisting of a human, a dog, a cat, a bovine, a pig, and a horse. In an especially preferred embodiment, the mammal is a human.

The methods of treating include administering immunogenic compositions comprising the polypeptides of the invention, but compositions comprising nucleic acids encoding these polypeptides as well. Those of skill in the art are cognizant of the concept, application, and effectiveness of nucleic acid vaccines (e.g., DNA vaccines) and nucleic acid vaccine technology as well as protein and polypeptide based technologies. The nucleic acid based technology allows the administration of nucleic acids encoding the polypeptides of the invention, naked or encapsulated, directly to tissues and cells without the need for production of encoded proteins prior to administration. The technology is based on the ability of these nucleic acids to be taken up by cells of the recipient organism and expressed to produce an immunogenic determinant to which the recipient's immune system responds. Typically, the expressed antigens are displayed on the surface of cells that have taken up and expressed the nucleic acids, but expression and export of the encoded antigens into the circulatory system of the recipient individual is also within the scope of the present invention. Such nucleic acid vaccine technology includes, but is not limited to, delivery of naked DNA and RNA and delivery of expression vectors encoding the polypeptides. Although the technology is termed “vaccine”, it is equally applicable to immunogenic compositions that do not result in a protective response. Such non-protection inducing compositions and methods are encompassed within the present invention.

Although it is within the present invention to deliver nucleic acids encoding the polypeptides of the invention and carrier molecules as naked nucleic acid, the present invention also encompasses delivery of nucleic acids as part of larger or more complex compositions. Included among these delivery systems are viruses, virus-like particles, or bacteria containing the nucleic acid encoding the polypeptides. Also, complexes of the invention's nucleic acids and carrier molecules with cell permeabilizing compounds, such as liposomes, are included within the scope of the invention. Other compounds, such as molecular vectors (EP 696,191, Samain et al.) and delivery systems for nucleic acid vaccines are known to the skilled artisan and exemplified in, for example, WO 93 06223 and WO 90 11092, U.S. Pat. No. 5,580,859, and U.S. Pat. No. 5,589,466 (Vical's patents), which are incorporated by reference herein, and can be made and used without undue or excessive experimentation.

To further achieve the objects and in accordance with the purposes of the present invention, a kit capable of diagnosing Leishmania infection is described. This kit, in one embodiment, contains the polypeptides of this invention. In another embodiment, the kit contains DNA sequences which encode the polypeptides of the invention, which are capable of hybridizing to Leishmania RNA or analogous DNA sequences to indicate the presence of a Leishmania infection. Different diagnostic techniques can be used which include, but are not limited to: (I) Southern blot procedures to identify cellular DNA which may or may not be digested with restriction enzymes; (2) Northern blot techniques to identify RNA extracted from cells; and (3) dot blot techniques, i.e., direct filtration of the sample through an ad hoc membrane, such as nitrocellulose or nylon, without previous separation on agarose gel. Suitable material for dot blot technique could be obtained from body fluids including, but not limited to, serum and plasma, supernatants from culture cells, or cytoplasmic extracts obtained after cell lysis and removal of membranes and nuclei of the cells by centrifugation.

Nucleic acid sequences within the scope of the invention include isolated DNA and RNA sequences that encode the polypeptides of the invention, sequences complementary to DNA and RNA sequences that encode the polypeptides of the invention, or sequences which hybridize to those DNA and RNA sequences under conditions of moderate or severe stringency. As used herein, conditions of moderate stringency, as known to those having ordinary skill in the art, and as defined by Sambrook et al. Molecular Cloning: A Laboratory Manual, 2 ed. Vol. 1, pp. 1.101-104, Cold Spring Harbor Laboratory Press, (1989), include, for example, use of a prewashing solution for the nitrocellulose filters 5×SSC, 0.5% SDS, 1.0 mM EDTA (pH 8.0), hybridization conditions of 50% formamide, 6×SSC at 42° C. (or other similar hybridization solution, such as Stark's solution, in 50% formamide at 42° C.), and washing conditions of about 60° C., 0.5×SSC, 0.1% SDS. Conditions of high stringency are defined as hybridization conditions as above, and with washing at 68° C., 0.2×SSC, 0.1% SDS. The skilled artisan will recognize that the temperature and wash solution salt concentration can be adjusted as necessary according to factors such as the length of the probe.

Recombinant expression vectors containing a nucleic acid sequence encoding the polypeptides of the invention can be prepared using well known methods. The expression vectors include DNA sequences operably linked to suitable transcriptional or translational regulatory nucleotide sequences, such as those derived from a mammalian, microbial, viral, or insect gene. Examples of regulatory sequences include transcriptional promoters, operators, or enhancers, an mRNA ribosomal binding site, and appropriate sequences which control transcription and translation initiation and termination. The ability to replicate in the desired host cells, usually conferred by an origin of replication, and a selection gene by which transformants are identified can additionally be incorporated into the expression vector.

In addition, sequences encoding appropriate signal peptides that are not naturally associated with the polypeptides of the invention can be incorporated into expression vectors. For example, a DNA sequence for a signal peptide (secretory leader) can be fused in-frame to a polypeptide of the invention so that the polypeptide is initially translated as a fusion protein comprising the signal peptide. A signal peptide that is functional in the intended host cells enhances extracellular secretion of the polypeptide of the invention. The signal peptide can be cleaved upon secretion of the polypeptide from the cell.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids. Commercially available vectors include those that are specifically designed for the expression of proteins. These include pMAL-p2 and pMAL-c2 vectors, which are used for the expression of proteins fused to maltose binding protein (New England Biolabs, Beverly, Mass., USA).

Promoter sequences commonly used for recombinant prokaryotic host cell expression vectors include β-lactamase (penicillinase), lactose promoter system (Chang et al., Nature 275:615, 1978; and Goeddel et al., Nature 281:544, 1979), tryptophan (trp) promoter system (Goeddel et al., Nucl. Acids Res. 8: 4057, 1980; and Eβ-A-36776), and tac promoter (Maniatis, Molecular Cloning: A Laboratory Manual, Cold Spring Harbor Laboratory, p. 412, 1982).

Suitable host cells for expression of the polypeptides of the invention include prokaryotes, yeast or higher eukaryotic cells. Appropriate cloning and expression vectors for use with bacterial, fungal, yeast, and mammalian cellular hosts are described, for example, in Pouwels et al. Cloning Vectors: A Laboratory Manual, Elsevier, New York, (1985). Cell-free translation systems could also be employed to produce these polypeptides using RNAs derived from DNA constructs disclosed herein.

This invention will be described in greater detail in the following Examples.

### EXAMPLE 1

### Materials and Methods

Sera. Nine sera from MVL patients strongly reacting with P32 were pooled in equal ratios (v/v) and designated as MVL sera pool to be used as a positive control. Ten sera from ZCL patients unreactive with P32 were also selected and pooled to be used as negative controls (ZCL sera pool).

Parasites. The antigens used in the study were prepared from a L. infantum isolate obtained from a Tunisian patient suffering from MVL (MHOM/TN87/KA412; Zymodeme MON-1). Promastigotes were grown at 26° C. in RPMI 1640 medium (Sigma, Germany) supplemented with 10% fetal calf serum and were harvested at the late log phase as previously described (61).

Membrane antigens (MBAs). MBAs were prepared from 10¹⁰ promastigotes (1 liter of culture). Cell pellets were washed and resuspended in 10 ml lysis buffer supplemented with protease inhibitors [LBi: 10 mM Tris-HCl pH 8, 2 mM ethylene diamine tetra-acetic acid (EDTA), 1 mM phenylmethylsulfonyl fluoride (PMSF), 2 mM ethylene glycol-bis (β aminoethyl ether)-N, N, N′, N′-tetra-acetic acid (EGTA), 2 μg/ml of pepstatin, 2,5 mM N-ethylmaleimide (NEM) and 0,127 IU/ml of aprotinin]. The lysate was maintained on ice for 20 min. The cell suspension was further disrupted by a Dounce homogenizer 60 strokes of pestel A (Kontes, New Jersey) and four cycles of sonifications of 10 sec (Vibra Cell Sonicator Sonics & Materials Inc Danbury Conn. USA). A first centrifugation was performed at 1500 g for 10 min at 4° C. to remove unbroken parasites and nuclei. The supernatant (S₁₅₀₀) containing the protein extracts was kept in ice. The pellet (P₁₅₀₀) was resuspended in 2 ml LBi and was then submitted as previously, to a second set of disruptions and centrifugation at 1500 g. The final pellet was discarded and the two supernatants (S₁₅₀₀) were pooled (total antigens, TAs) and centrifuged at 10,000 g for 30 min at 4° C. to generate a supernatant (soluble antigens, SAs) and a pellet fraction (membrane antigens, MBAs). The P_{10,000} pellet, containing the crude membrane antigens, was resuspended in 5 ml of TBS (10 mM Tris-HCl pH 7.4, 150 mM NaCl) supplemented with protease inhibitors as described above. Aliquots (0.5 mg/0.5 ml), of the crude membrane antigen preparation, were conserved at −80° C. until use.

To obtain integral membrane antigens, one aliquot (500 μg) of crude membrane antigens (MBAs) was centrifuged for 10 min at 4° C., at 2,000 g. The pellet was resuspended in 1 ml of (10 mM Tris-HCl pH 11, 2 mM EDTA, 30% sucrose) and then incubated at +4° C. under agitation for 1 h. After one freeze/thaw cycle, the L. infantum integral membranes were purified by ultracentrifugation at 90,000 g, for 4 h at 4° C., in a Beckman L7 ultracentrifuge using SW₂₅₋₁ rotor. The peripheral membrane antigens (PMAs) were collected in the supernatant (S_{90,000}) and the integral membrane antigens (IMAs) were sedimented in the pellet (P_{90,000}). Protein yields. were estimated by the Bradford assay (9) and the fractions obtained at two different centrifugation steps (at 10,000 and 90,000 g) were analyzed by immunoblot, of one-dimensional SDS-PAGE, with the MVL sera pool.

SDS-PAGE. Sodium dodecyl sulfate-polyacrylamide gel electrophoresis (SDS-PAGE) was performed under denaturing and reducing conditions using a 15% acrylamide-bisacrylamide (29:1) gel as described by Laemmli (23). Two vertical electrophoresis systems were used: (i) mini-system (8.5×6.5×1 mm thickness, Mini Protean II, Bio-Rad) for analyses purposes and (ii) standard-size electrophoresis apparatus (14×16 cm×1.5 mm thickness, LKB instruments) for preparative purposes. The gels were stained with Coomassie blue G (Gold) (Fluka; France 0.25%; w/v) or electroblotted.

The molecular mass standards (Bio-Rad Laboratories, Calif., USA) used corresponded to phosphorylase b (97,400 kDa), serum albumin (BSA, 66,200 kDa), ovalbumin (45 kDa), carbonic anhydrase (31 kDa), trypsin inhibitor (21,500 kDa) and lysozyme (14,400 kDa).

Immunoblofting. Once the 1 or 2D-electrophoresis were finalized, the polyacrylamide gels were electro-transferred onto a 0.45 μm-pore-size polyvinylidene difluoride (PVDF) membranes, (Amersham, France), for 2 h 30 min at 50 V and at +4° C., using 10 mM (3-[Cyclohexylamino]-1-propanesulfonic acid) CAPS, pH 11, containing 10% methanol as transfer buffer, according to Matsudaira (34) without any membranes staining. The membranes were then incubated for 2 h at room temperature under agitation in [PBS-Tween 20-Milk (1X-1%-5%)] with the MVL sera pool diluted at 1/10000 (SDS-PAGE) or at 1/2000 (2D-PAGE), respectively, with the ZCL sera pool diluted at 1/200, and with the polyclonal anti-peptide sera diluted at 1/1000 or at 1/20000 (2D-PAGE). After 3 washes in PBS-Tween, the different Ag-Ab complexes were detected after incubation with peroxidase- conjugated anti-human sheep or anti-rabbit donkey immunoglobulins (Amersham International Inc., United Kingdom) diluted at 1/1000 and 1/2000 in PBS-Tween, respectively. The peroxidase enzymatic activity was revealed by incubation in 0.05% 3-3,′-diaminobenzidine; (DAB; Sigma) and 0.03% H₂O₂ in 50 mM Tris-HCl pH 7.6. In the case of 2D-PAGE, the proteins were detected by enhanced chemiluminescence (ECL) assay, according to the supplier's instructions (Amersham, France), at different exposure times.

Detergent solubilization of the antigenic P32 fraction. To further determine the nature of the association of antigenic P32 fraction with membranes, and after treatment at pH 11, equal aliquots of the pellet P_{90,000} (100 μg) were resuspended in 1 ml of PBS pH 7.2 containing one of the following detergents: 1% TritonX-100 (TX-100), 1% Digitonin (Dig), 1% 3-[(3-Cholamidopropyl)dimethylamino]-17-propanesulfonate (CHAPS), 1% Sodium Cholate (Na Cholate), 1% Octyl-β-D-glucopyranoside (OG) or 0.5% Lauryidimethylamine oxide (LDAO). All samples were incubated for 1 h at 4° C. under vigorous agitation and were centrifuged for 1 h at 90,000 g, in a Beckman L7 ultracentrifuge using SW₂₅₋₁ rotor, at 4° C. The supernatants were submitted to electrophoresis and subsequently immunoblotted.

Peptide Synthesis. The two prominent bands (P32 and P33 kDa) revealed by Coomassie blue and reactive with MVL sera but not ZCL sera, were cut apart from six preparative SDS-PAGE gels and digested, with sequencing grade lysine-C protease (Boehringer Mannheim) and trypsine (Sigma, Germany) in Tris-HCl 100 mM, pH 8.8 with 0.003% SDS or 0.01% Tween 20 (w/v), respectively. The enzyme/protein ratios were those recommended by the supplier's instructions. The two mixtures were incubated for 18 h at 30° C. The peptides generated from the P32 and P33 bands, were purified by C18-DEAE HPLC column using a linear gradient of 2-35% and 2-45% acetonitril in 0.1% trifluroacetic acid (TFA) over40 min, respectively. Four different peptides: P1 and P2 and P3 and P4 containing 12, 15, 12 and 9 residues, deriving from the enzymatic digestion of the P32 and P33 bands respectively, were selected for amino acid sequencing, on the basis of peak homogeneity and prominence. The peptides were sequenced using an Applied Biosystems apparatus ABI 473. The four peptides sequenced were then chemically assembled by the solid-phase method (35) using a peptide synthesizer (model 433A; Applied Biosystem, Foster City, Calif.). Stepwise elongation of the peptide chain was carried on a 4-hydroxymethyl-phenoxymethyl resin (0.96 me/g) using optimized fluorenylmethyloxycarbonyl (Fmoc)/ter-butyl strategy. The Fmoc-amino acid derivatives were coupled as their hydroxybenzotriazole active esters in N-methylpyrrolidone. After trifluoroacetic acid cleavage, the crude peptides were purified by C18 reversed phase HPLC. The purified peptides were further characterized by: (i) analytical C18 reversed phase HPLC, (ii) amino acid analysis after acidolysis, and (iii) mass determination by matrix assisted laser desorption ionization-time of flight mass spectrometry.

Peptide-KLH conjugation and Rabbit immunization. The purified peptides were conjugated to keyhole limpet hemocyanin (KLH; Sigma, Germany.) through a N-terminal α-amino group using an homobifunctional reagent glutharaldehyde (GA; Sigma, Germany) by the procedure described by Pfaff et al. (45).

The conjugated peptides (250 μg/0.5 ml) were mixed with complete Freund's adjuvant at a 1:1 ratio and inoculated intradermally to rabbits. Three booster doses were administrated at days 30, 60 and 90 using subcutaneous injection of the antigen admixed with incomplete Freund's adjuvant (v/v). Rabbits were bled before immunization and ten days after each boost. Antibody titers were defined by ELISA.

Enzyme- linked immunosorbent assay (ELISA.). Rabbit antisera to P32 or P33 derived synthetic peptides and pooled MVL sera were checked for their ability to react with the immunizing peptides, by an indirect ELISA according to Voller et al. (62) and Pffaf et al. (45) with some modifications. Polystyrene 96-well plates (Nunc) were coated overnight at +4° C. or for two hours at 37° C. with 50 μl of synthetic peptides (10 μg/ml) diluted in phosphate-buffered saline (PBS: 10 mM, pH 7.4). Excess coating buffer was flicked off and non-specific binding sites were blocked with 5% skimmed milk in PBS containing 0.1% Tween 20 (PBS-T-M) for 1 h at 37° C. After three washes with 0.1% Tween 20 in PBS (PBS-T), the plates were incubated for 2 h at 37° C. in presence of 50 μl anti-peptide antisera, taken from the different rabbit sera (dilutions from 1/100 and up to 1:10⁵) or MVL sera pool (diluted at 1/100 in PBS-T-M). Unbound antibodies were washed off five times as above, and peroxidase-conjugated donkey anti-rabbit or sheep anti-human immunoglobulin (Amersham Life Science International plc, England U.K) diluted at 1/2000 and at 1/1000 in PBS-T respectively, were added and followed by incubation for 1 h at 37° C. Unbound conjugate was washed off six times, and 100 μl of orthophenylene diamine (Sigma, Germany) (1 mg/ml, w/v) dissolved in citrate buffer 100 mM, pH 5.0 containing 0.03% (v/v) hydrogen peroxide (H₂O₂) were subsequently added. The plates were incubated for 20 min at room temperature in the dark, and the reactions were stopped by addition of 50 μl per well of a 4N sulphuric acid solution (H₂SO₄). The optical density was measured at 492 nm in an ELISA reader (Titerteck-multiskan, Helsinki, Finland), and titers were defined when necessary.

Two-dimensional electrophoresis (2D-PAGE). Two-dimensional NEPHGE/SDS-PAGE was performed according to O'Farell et al. (39) with minor modifications (43). Briefly, proteins were separated in the first dimension using two ampholytes ratio: 1:1:2 or 1:3 (pH 3/10-pH 5/7-pH 6/8) respectively. The run was carried out in the opposite direction as compared to the typical isoelectric focusing gel (IEF). Phosphoric acid (0.011 M H₃PO₄) was placed in the upper chamber and NaOH (0.1 M) solution in the lower one. The connections to the power supply were also reversed. The optimised migration conditions corresponded to: 200 V for 30 min then 300 V for 30 min and 400 V for 6 H, at 20° C. The second dimension (SDS-PAGE) was performed as described above.

The protein concentration was evaluated by Bradford's protein assay (9) modified by Ramagli et Rodriguez (50).

Analysis of 2D-polypeptide pafterns. For sake of reproducibility between successive migrations, equivalent amounts of the L. infantum promastigote membrane antigens were run under identical conditions. Immunoblots revealed by the MVL sera pool were compared to Coomassie blue stained-gels by visual estimation of the relative intensities and positions of the spots corresponding to each polypeptide. The immunodominant antigens corresponding to abundant spots were identified on the gels and were considered for further analysis. Similarly, immunoblots of MAs separated in the narrow pH range (1 part pH 3/10 and 3 part pH 6/8) NEPHGE/2D-PAGE, revealed by polyclonal rabbit sera anti-P1, anti-P2 or anti-P3 peptides, were compared to immunoblots revealed by MVL sera pool.

Protein Identification by Liquid Chromatography Mass Spectrometry (LC-MS/MS). Membranes antigens (MBAs) were separated by NEPHGE/2D-PAGE as described above. Six preparative gels were stained by Coomassie blue. Visible polypeptides of interest in the 30-36 kDa area were excised from gels and stored in 100 μl of HPLC-grade water at 4° C. until subsequent digestion and LC/MS-MS analysis. The gel digestion procedure was carried out as described by Rabilloud et al. (49).

Mass Spectrometry. The MS and MS/MS mass measurement were performed with a Q-TOF 2 hybrid quadrupole/ time-of-flight mass spectrometer (Micromass Ltd., Manchester, UK) equipped with a Z-spray ion source and the liquid junction. The instrument consists of an electrospray ionization source, a quadrupole mass filter operating as a variable bandpass device, an hexapole collision cell, and an orthogonal acceleration time-of-flight (TOF) mass analyzer. The TOF mass analyzer is used to acquire data both in MS and MS/MS modes.

Nano LC/MS/MS data was collected using data-dependent scanning, that is, automated MS to MS/MS switching. The data-dependent scanning used, is one collision energy for each precursor, with the collision energy used based on the charge state and the m/z of the precursor ion. The spray system (liquid junction) was at 3.5 kV.

Data Processing and data analysis. Data processing of LC/MS/MS data was done automatically with the ProteinLynx Process (MicroMass) module. Data analysis was done with Global Server (MicroMass, Ltd., Manchester, UK) software and Mascot (Matrix Science Ltd., London, UK) against the NCBlnr data base.

Nano HPLC. A CapLC (Micromass Ltd., Manchester, UK) system was used for sample injection and pre-concentration. The sample pre-concentration and desalting were done on a pre-column cartridge packed with a 5 μm 100 Å C18 PepMap stationary phase (LC-Packings) with length of 1 mm and an ID of 300 μm at 30 μl/min during 3 min. The loading solvent for sample pre-concentration and clean-up consisted of 0.1% formic acid in water.

After clean-up, the pre-concentration system is switched (Stream Select) and the pre-column placed on in-line with the analytical column. Bound peptides are back-flushed eluted from the pre-column onto the analytical column. Peptides were separated on a 15 cm×75 μm ID column, packed with 3 μm 100 A Å C18 PepMap (LC-Packings) stationary phase. Mobile phase A consisted of 0.1% formic acid in water and mobile phase B of 0.1% formic acid in acetonitrile. The elution was performed at a flow rate of 200 nl/min, a 545% gradient (mobile phase B) over 35 min, followed a 95% (solvent B) over 5 min and the re-equilibration of the column is done during 20 min by 100% of mobile phase A.

Bioinformatic analyses. The sequences generated through this invention were submitted to search for homologies using the BLAST servers of the NCBI or of the Leishmania genome project (www.sanger.ac.uk./projects/L_major). Alignments were generated using the Clustal W program (www.ebi.ac.uk/clustalw). Data relating to Leishmania genes/products were extracted from the L. major gene data base (gene DB, www.genedb.org/genedb/Leish/index.jsp) or from the NCBI and Swiss-Prot databases (www.ncbi.nlm.nih.gov.;us.expasy.org/Sprot).

### EXAMPLE 2

### Enrichment and Solubilization of Membrane Associated 30-36 kDa Leishmania infantum Antigens

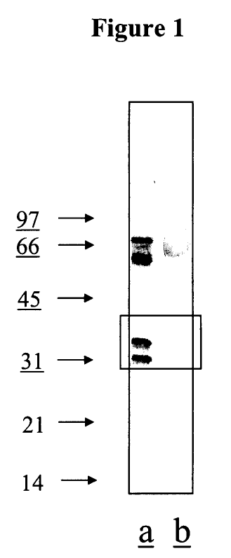

Previously, it was shown, using western blots of membrane antigens of L. infantum parasites, that a P32 kDa antigen(s) was recognized by 95% of MVL sera, but not by ZCL sera. In order to characterize further this antigenic fraction, 9 sera from MVL patients were selected and 10 sera from ZCL patients. They were characterized individually for their reactivity to the Leishmania MBAs and were then pooled and tested. All MVL sera reacted with bands at 32 and 33 kDa, and to a lesser extent with bands at 30 kDa (7/9) and 36 kDa (4/9). The reactivity of the pooled MVL sera was representative of the individual ones (FIG. 1a). As expected, the 10 ZCL sera and their pool did not react with the 30-36 kDa region, as this pool was considered the negative control sera in this study (FIG. 1b). Given the hypothesis that the antigenic fraction of interest is constituted by different proteins, the pooled MVL sera was considered as a good tool to analyse the reactivity of different patients' sera.

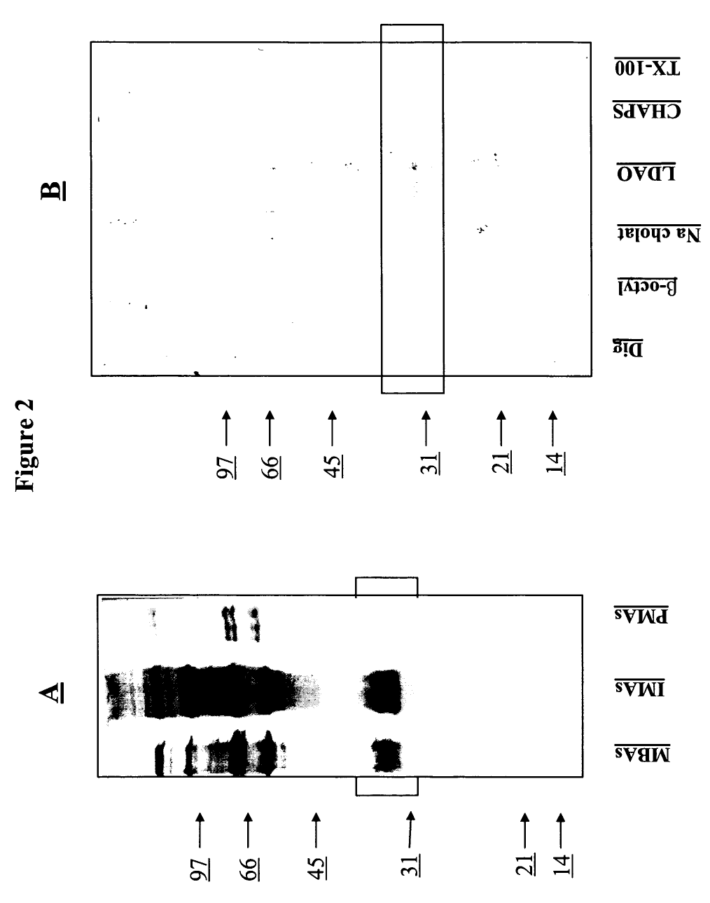

The association of the antigens to cell membranes was determined. In order to enrich the crude membrane preparations for the 30-36 kDa antigens recognized by the MVL sera pool, and to solubilize them, MBA extracts were submitted to different treatments and were analysed by one-dimensional SDS-PAGE and immunoblotting. To remove peripheral proteins, the MBAs were first treated at pH 11. As shown in FIG. 2, alkaline treatment did not extract the bulk of reactive antigens, except for a partial solubilization of the p36 kDa. This suggests that the most reactive components could be considered as integral membrane proteins (FIG. 2A). The insoluble fraction obtained after incubation at pH 11 was further treated with either one of the following detergents: (LDAO) (0.5%), Na cholate (1%), OG (1%), Digitonin (1%), T-X 100 (1%) or CHAPS (1%). As illustrated in FIG. 2B, only LDAO partially solubilized P32 antigen(s).

These results indicate that the P32 kDa band has a heterogeneous composition and that some of the antigens in the area of 30-36 kDa are either integral proteins or are strongly complexed to the membranes. Furthermore, one could not exclude the occurrence of isoforms within each individual band migrating at a given electrophoretic position. The difficulties in enriching for these antigens or solubilizing them led to the use of alternative approaches to characterize the 30-36 kDa antigens.

### EXAMPLE 3

### Microsequencing Analysis of the 32 and 33 kDa Bands.

The 32 and 33 kDa protein bands resolved on SDS-PAGE gels, were stained with Coomassie blue, then cut out from the gel and digested using lysine-C protease and trypsin, respectively. Four peptides, P1 and P2 from 32 kDa band, and P3 and P4 from 33 kDa band, were selected for sequencing. Peptides sequences were as follows: P1 (KLLVQNQGEMIK) [SEQ ID NO: 1], P2 (KAPSEWMGGVM/GFVNK) [SEQ ID NO: 2], P3: (KLGQGISLIMIK) [SEQ ID NO: 3] and P4: (KDLVPLWGR) [SEQ ID NO: 4].

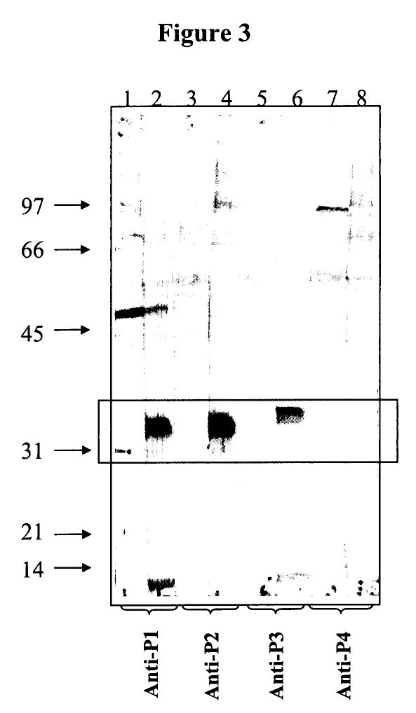

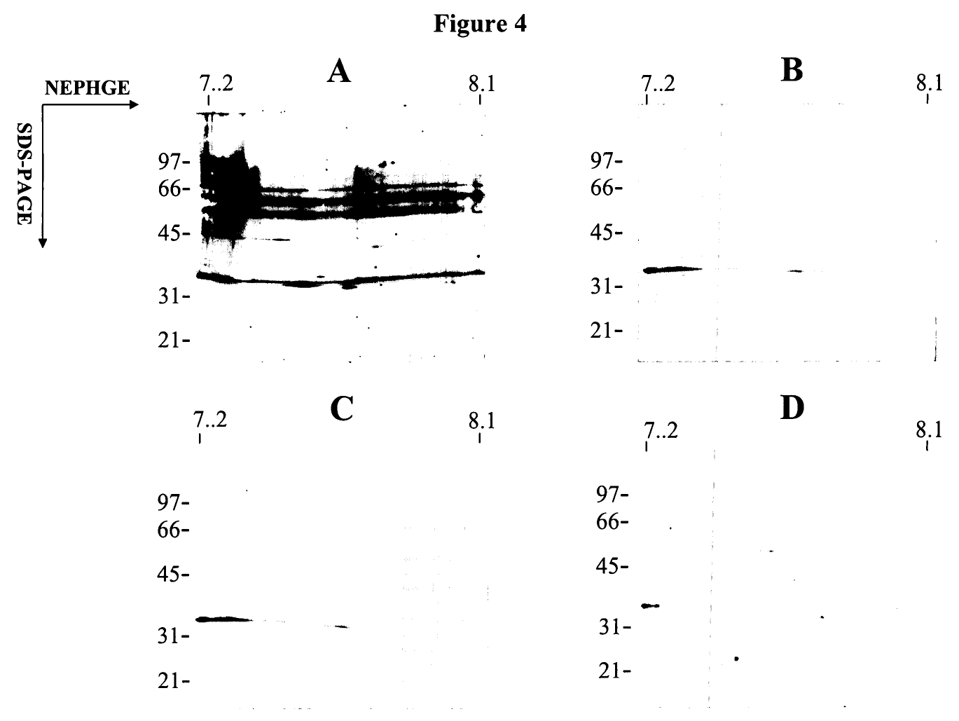

Based on these sequences, the four peptides were chemically synthesized, coupled to KLH as a carrier, and then inoculated to rabbits to produce polyclonal anti-sera. The resulting anti-sera were tested by immunoblotting on MBAs (FIG. 3). The anti-P1 and anti-P2 sera strongly reacted with a major band at 33 kDa, and to a lesser extent with an additional band at 30 kDa. Anti-P3 sera specifically reacted with a 35 kDa band. Anti-P4 sera did not react with any membrane protein in the range of 30-36 kDa MM, and was not further analysed. These results were confirmed by immunoblots of 2D gels (FIG. 4). As shown in FIG. 4, each of the polyclonal sera anti-P1, anti-P2 and anti-P3 reacted very specifically with proteins migrating within the pH range of 7.2-8.10 at the sizes observed on 1D gels, 33, 33 and 35 kDa, respectively. It seems clear that each of the patterns observed contribute to the reactivity of the MVL sera pool in the 30-36 kDa area. However, reactivity of anti-P1 and anti-P2 appeared stronger than the reactivity of anti-P3. Furthermore, each of the P1, P2 and P3 peptides tested by ELISA, strongly and specifically reacted with MVL patients sera as compared to ZCL and normal human sera (data not shown).

Finally, the sequences of P1, P2 and P3 peptides were analysed using the regular BLAST and the “search for short nearly exact matches” BLAST option, on the NCBI nr database. Significant matches with known products were observed with P1 and P3, when using this last option. P1 presented a 100% identity with a NH₂-terminal sequence (residues 48-59: KLLVQNQGEMIK [SEQ ID NO: 1) of L. major mitochondrial ADP/ATP carrier proteins (Q9N674 and Q9N647), which have a predicted molecular mass of 35 kDa and an isoelectric point of 10.1.

Peptide (P3) presented a 83% identity with a T. brucei brucei protein (residues 274-285: KLGQGISLIMIK [SEQ ID NO: 3]) of 37 kDa corresponding to the glycosomal glycerol-3-phosphate dehydrogenase (P90593). However, the alignment of the last peptide to the glycerol 3-phosphate dehydrogenase proteins described for T. brucei brucei (P90593), L. major (LmjF10.0510), L. mexicana (P90551) and T. brucei rhodesiensis (Q26756) did not confirm that P3 corresponds to a peptide deriving from the glycerol 3 phosphate dehydrogenase protein from L. infantum. Submission of the sequence of the 3 peptides to the Omniblast server of L. major genome project confirmed that P1 is actually part of the ADP/ATP carrier protein (100% identity, E=2e-005). Peptide P2 showed a 93% identity, (E=2e-006) with residues 152-166 of a putative NADH-cytochrome b5 reductase protein, which has an expected MM of 32.290 kDa and a PI of 8.55. Residues 2-10 of P3 presented 100% identity, (E=0.0015) with residues 62-70 of a predicted putative mitochondrial carrier protein, which has an expected MM of 34.661 and a PI of 9.5. These three proteins are most likely localized in the inner mitochondrial membrane, and would correspond to integral membrane antigens.

The significant e-value observed for these hits together with the percentage of identity, the concordance of the protein sizes observed with the predicted ones and the comparable reactivity of the anti-peptides to the MVL sera, led to the conclusion that mitochondrial carrier proteins and cytochrome b5 reductase proteins act as targets of the humoral immune responses during MVL. Furthermore, these results confirm the initial hypothesis about the heterogeneous content of the 30-36 kDa antigenic fraction.

### EXAMPLE 4

### NEPHGE-2D Analysis of the 30-36 kDa Antigens Reacting with the MVL Sera Pool

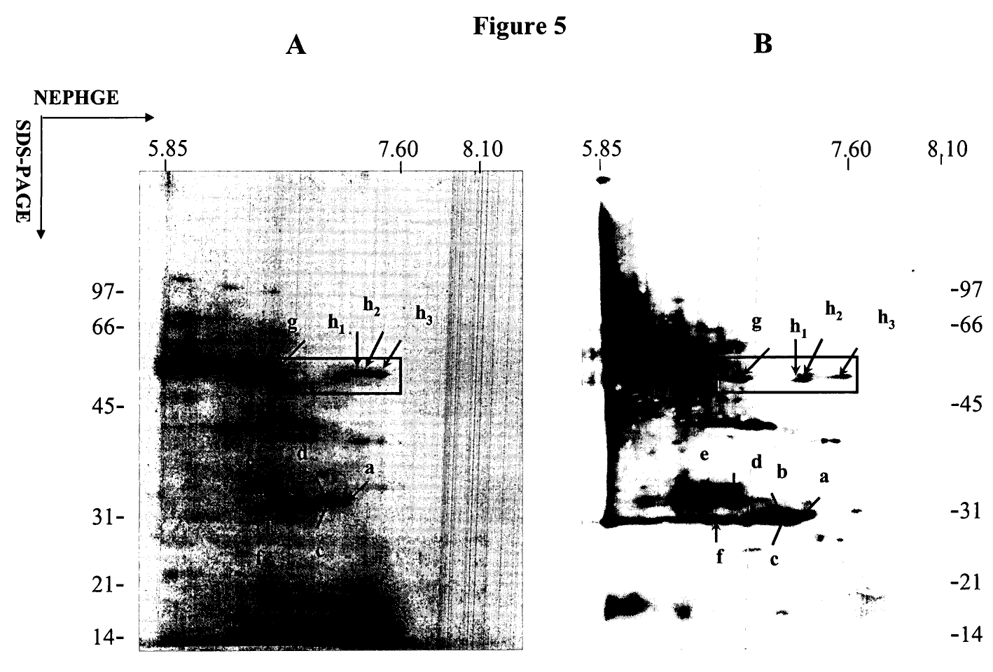

In order to further resolve the components of membrane associated antigens migrating by 1D-electrophoresis in the 30-36 kDa MM range, MBA preparations were submitted to NEPHGE-2D in the pH interval of 5.85-8.10, followed by SDS-PAGE analysis. Several gels were run in parallel, and were either stained with Coomassie blue or transferred onto PVDF membranes to be incubated with the MVL sera pool. At least 14 polypeptides, in the size range of interest, resolving within a pH range of 5.85-7.6 reacted with the MVL sera pool but not with the ZCL sera pool. Among them, 6 spots were also detectable on Coomassie blue stained gels, and were considered to be present in suitable amounts for further analysis using LC-MS/MS (FIG. 5).

### EXAMPLE 5

### Molecular Characterization of the 30-36 kDa Antigens Reactive With MVL Sera Pool, by LC-MS-MS Analysis

The six immunodominant and abundant spots revealed by NEPHGE-2D analysis were excised from gels and submitted to LC-MS-MS analysis. The mass spectra obtained from each spot provided sufficient signal to search databases using the ProteinLynx Process. For all spots, significant hits were observed providing a sequence coverage ranging from 8 to 23% (Table 1). The results were further confirmed by the sequence analysis of a selection of the matching peptides.

TABLE 1
Summary of LC-MS/MS analysis of 10 Leishmania infantum promastigote membrane antigens spots,
recognized by pooled MVL sera on Western blots of two-dimensional NEPHGE-SDS-PAGE.

| | | | | | Swiss-Prot | | |
|---|---|---|---|---|---|---|---|
| | pH at end | Apparent | | Hits | Accession | Probability | Sequence |
| | of | MM | | MM/PI | Number | Based | coverage |
| Spots | migration | (kDa) | Protein/organism | kDa | (AN) | Mowse Score* | (%) |
| a | 7.4 | 32 | Elongation factor 1-alpha (L. donovani) | 49.097/9.03 | Q95VF2 | 140 | 8 |
| b | 7.3 | 32 | Elongation factor 1-alpha (L. donovani) | 49.097/9.03 | Q95VF2 | 315 | 23 |
| c | 7.3 | 31.5 | Elongation factor 1-alpha (L. donovani) | 49.097/9.03 | Q95VF2 | 145 | 9 |
| d | 6.95 | 34 | Guanine nucleotide binding protein | 34.351/6.05 | Q27434 | 195 | 21 |
| | | | beta subunit-like activated protein | | | | |
| | | | kinase C receptor homolog LACK. | | | | |
| | | | (L. donovani | | | | |
| | | | Probable aldehyde reductase EC | 31.830/5.70 | P22045 | 115 | 20 |
| | | | 1.1.1 —). (L. major) | | | | |
| e | 6.7 | 33 | Probable ubiquinol-cytochrome-c | 33.634/6.03 | Q27785 | 155 | 17 |
| | | | reductase (EC 1.10.2.2) Rieske iron- | | | | |
| | | | sulfur protein precursor. (Trypanosoma | | | | |
| | | | brucei). | | | | |
| f | 6.8 | 31 | Guanine nucleotide binding protein | 34.351/6.05 | Q27434 | 160 | 20 |
| | | | beta subunit-like activated protein | | | | |
| | | | kinase C receptor homolog LACK. | | | | |
| | | | (L. donovani). | | | | |
| | | | Probable aldehyde reductase (L. major). | 31.816/5.70 | P22045 | 160 | 12 |
| g | 6.9 | 50 | Elongation factor 1-alpha (L. donovani) | 49.097/9.03 | Q95VF2 | 90 | 21 |
| h₁ | 7.45 | 50 | Elongation factor 1-alpha (L. donovani) | 49.097/9.03 | Q95VF2 | 725 | 53 |
| h₂ | 7.5 | 50 | Elongation factor 1-alpha (L. donovani) | 49.097/9.03 | Q95VF2 | 550 | 34 |
| h₃ | 7.6 | 50 | Elongation factor 1-alpha (L. donovani) | 49.097/9.03 | Q95VF2 | 300 | 22 |

*Score is −Log * (P), where P is the probability that the observed match is a random event.

For the six selected spots, four Leishmania proteins were unambiguously identified, which correspond to products functionally associated to parasite cell membranes, some of which belonging to the glycosomal or the mitochondrial compartments. For spots d and f, of 34 and 31 kDa MM, respectively, two proteins were concomitantly identified within each spot as guanine nucleotide binding protein beta subunit-like known as the activated protein kinase C receptor homolog LACK antigen (17) and a probable aldehyde reductase (55). Spot e with a Mowse score of 155 and a sequence coverage of 17%, corresponded to a putative T. brucei reiske iron-sulfur protein precursor (EC 1.10.2.2.), which is imported to the mitochondrion to be part of the ubiquinol-cytochrome-c reductase complex (46). The search for a homolog in Leishmania gene data base allowed to identifying a product encoded by chromosome 35. This protein bears 297 residues and has 84.5% identity with the T. brucei product, and has expected MM and PI of 33.634 kDa and 6.03, respectively. The matching peptides of spot e mapped on identical parts of the proteins from both organisms, confirming the identification of this spot.

In the case of the polypeptides a, b, and c, having apparent molecular masses of 32, 32 and 31.5 kDa, respectively, the LC-MS/MS analysis provided as significant hits the elongation factor 1 alpha from L. donovani promastigotes. This protein is actually described as being a highly abundant protein in eukaryotic cells, having a 49-50 kDa MM (38), a size significantly higher than the 32 kDa characterized by this invention. This prompted a check that the elongation factor in these preparations is also identified at its expected size (49-50 kDa) and that it is reactive with the MVL sera. Four spots (g, h₁, h₂ and h₃), strongly reacting with the MVL sera pool within the 49-50 kDa range with a pH at end of migration of 6.9, 7.45, 7.5, and 7.6, respectively, closer to the expected PI of 9.03 than the other spots at this size, were selected for LC-MS/MS analysis (FIG. 5). As expected, these spots were identified as elongation factor 1 alpha. The matching peptides identified by LC-MS/MS were aligned to their corresponding Leishmania counterparts. Interestingly, peptides of spots d, e, f, g, h₁, h₂ and h₃ matched sequences, which were located at either the NH₂, central or the COOH terminal parts of the corresponding Leishmania proteins (data not shown). However, spots a, b, and c provided peptides, which matched only the N- terminal part of the elongation factor 1 alpha (FIG. 5). The fact that the latter polypeptides had consistently smaller molecular mass than expected, suggested that spots a, b, and c corresponded to truncated elongation factors. Consultation of the gene database (gene DB) specific to Leishmania major have confirmed this hypothesis. Indeed, besides the gene coding for 49-50 kDa elongation factor 1-alpha, the Leishmania genome project uncovered on the chromosome 19, several other genes coding for truncated elongation factors-1α, which products have expected sizes at 36.4, 34.5 and 30.6 kDa, corresponding to various lengths of the N-terminal part of the protein.

In summary, six immunodominant Leishmania products involved in the humoral response of MVL patients, and unreative with ZCL sera, have been identified. These 6 proteins are part of the 30-36 kDa complex that strongly reacts in Western blots with MVL sera.

### REFERENCES

The entire disclosures of each of the following publications are relied upon and incorporated by reference herein.

1. Anonymous. 1990. Control of leishmaniases. Report of a WHO Expert Committee. Word Health Organ. Tech. Rep. Ser. 793:1-158.

2. Badaro, R., D. Benson, M. C. Eulalio, M. Freire, S. Cunha, E. M. Netto, D. Pedral-Sampaio, C. Madureira, J. M. Burns, R. L. Houghton, J. R. David, and S. G. Reed. 1996. rK39: a cloned antigen of Leishmania chagasi that predicts active visceral leishmaniasis. J. Infect. Dis. 173:758-761.

3. Badaro, R., S. G. Reed, A. Barral, G. Orge, and T. C. Jones. 1986. Evaluation of the micro enzyme-linked immunosorbent assay (ELISA) for antibodies in American visceral leishmaniasis; antigen selection for detection of infection-specific responses. Am. J. Trop. Med. Hyg. 35:72-78.

4. Badaro, R., S. G. Reed, and E. M. Carvalho. 1983. Immunofluorescent antibody test in American visceral leishmaniais: sensitivity and specificity of different morphological forms of two Leishmania species. Am. J. Trop. Med. Hyg. 32:480-484.

5. Ben Salah, A., R. Ben-Ismail, F. Amri, S. Chlif, F. Ben Rzig, H. Kharrat, H. Hadhri, M. Hassouna, and K. Dellagi. 2000. Investigation of the spread of human visceral leishmaniasis in central Tunisia. Trans. R. Soc. Trop. Med. Hyg. 94:382-386.

6. Bente, M., S. Harder, M. Wiesgigl, J. Heukeshoven, C. Gelhaus, E. Krause, J. Clos, and I. Bruchhaus. 2003.Developmentally induced changes of the proteome in the protozoan parasite Leishmania donovani. Proteomic. 3:1811-1829.

7. Berneman, A., X. Rolland, and G. Broun. 1988. Humoral response in Leishmania infantum clinical infections. Ann. Inst. Pasteur Immunol. 139:267-278.

8. Bourreau, E., G. Prevot, J. Gardon, R. Pradinaud, H. Hasagewa, G. Milon, and P. Launois. 2002. LACK-specific CD4(+) T cells that induce gamma interferon production in patients with localized cutaneous leishmaniasis during an early stage of infection. Infect. Immun. 70:3122-3129.

9. Bradford, M. 1976. A rapid and sensitive method for the quantification of microgram quantities of protein utilizing the principle of protein-dye binding. Anal. Biochem. 72:248-254.

10. Bray, R. S. 1976. Immunodiagnosis of leishmaniasis, p. 66-76. In S. Cohen, and E. H Sadun, (eds), Manual of immunology of parasitic infections, Blackwell Scientific Publications, London.

11. Burns, J. M. Jr, W. G. Shreffler, D. R. Benson, H. W. Ghalib, R. Badaro, and S. G. Reed. 1993. Molecular characterization of a kinesisn-related antigen of Leishmania chagasi that detects specific antibody in African and American visceral leishmaniasis. Proc. Natl. Acad. Sci. USA. 90:775-779.

12. Chulay, J. D., and A. D. M. Bryceson. 1983. Quantification of amastigotes of Leishmania donovani in smears of splenic asperates from patients with visceral leishmaniasis. Am. J. Trop. Med. Hyg. 32:475-479.

13. Evans, T. G., E. C. Krug, M. E. Wilson, A. W. Vasconcelos, J. E. De Alencar, and R. D. Pearson. 1989. Evaluation of antibody responses in American visceral leishmaniasis by ELISA and immunoblot. Mem. Inst. Oswaldo Cruz, Rio de Janeiro 84:157-166.

14. Fargeas, C., M. Hommel, R. Maignon, C. Dourado, M. Monsigny, and R. Mayer. 1996. Synthetic peptide-based enzyme-linked immunosorbent assay for serodiagnosis of visceral leishmaniasis. J. Clin. Microbiol. 34:241-248.

15. Flinn H. M., D. Rangarajan, and D. F. Smith. 1994. Expression of hydrophilic surface protein in infective stages of Leishmania major. Mol. Biochem. Parasitol. 65:259-270.

16. Ghedin, E., W. W. Zhan, H. Charest., S. Sundar, R. T. Kenney and G. Matlashewski. 1997. Antibody Response against a Leishmania donovani Amastigote-Stage-Specific Protein in Patients with Visceral leishmaniasis. Clin. Diag. Labo. Immunol. 4:530-535.

17. Gonzalez-Aseguinolaza, G., S. Taladriz, A. Marquet, and V. Larraga. 1999. Molecular cloning, cell localization and binding affinity to DNA replication proteins of the p36/LACK protective antigen from Leishmania infantum. Eur. J. Biochem. 259:909-916.

18. Handman E, A. H. Osborn, F. Symons, R. van Driel, and R. Cappai. 1995. The Leishmania promastigote surface antigen 2 complex is differentially expressed during the parasite life cycle. Mol. Biochem. Parasitol. 74:189-200.

19. Harith, A. E., A. H. J. Kolk,. P. A. Kager., J. Leeuwenburg, R. Muigai, S. Kiugu, S. Kiugu, and J. J. Laarman. 1986. A simple and economical direct agglutination test for serodiagnosis and seroepidemiological studies of visceral leishmaniasis. Trans. R. Soc. Trop. Med. Hyg. 80:583-587.

20. Hayshi, Y., R. Urade, S. Utsumi, and M. Kito. 1989. Anchoring of a peptide elongation factor EF-1α by phosphatidylinositol at the endoplasmic reticulum membrane. J. Biochem. 106:560-563.

21. Ho L. C, A. Armiugam, K. Jeyaseelan, E. H. Yap, and M. Singh. 2000. Blastocystis elongation factor-1alpha: genomic organization, taxonomy and phylogenetic relationships. Parasitology. 121:135-144.

22. Hoerauf, A., P. Andrade, C. R. Andrade, W. Solbach, and M. Rollinghoff. 1992. Immunoblotting as a valuable tool to differentiate human visceral leishmaniasis from lymphoproliferative disorders and other clinically similar diseases. Res. Immunol. 143:375-383.

23. Hommel, M., W. Peters, J. Ranque, M. Quilici, and G. Lanotte. 1978. The micro-ELISA technique in the serodiagnosis of visceral leishmaniasis. Ann. Trop. Med. Parasitol. 72:213-218.

24. Jaffe, C. L., and M. Zalis. 1988. Purification of two Leishmania donovani membrane proteins recognized by sera from patients with visceral leishmaniasis. Mol. Biochem. Parasitol. 27:53-62.

25. Jensen, A. T. R., K. Kemp, T. G. Theander, and E. Handman. 2001. Cloning, expression and antigenicity of the L. donovani reductase. APMIS 109:461-468.

26. Kar, K. 1995. Serodiagnosis of leishmaniasis. Crit Rev Microbiol. 21:123-152.

27. Kaur, K. J., and L. Ruben, 1994. Protein translation elongation factor 1α from Trypanosoma brucei binds calmodulin. J. Biol. Chem. 269:23045-23050.

28. Laemmli, U. K. 1970. Cleavage of structural proteins during the assembly of the head of bacteriophage T4. Nature. 227:680-685.

29. Liu G., B. T. Edmonds, and J. Condeelis. 1996. pH, EF-1alpha and the cytoskeleton. Trends Cell. Biol. 6:168-171.

30. Louzir H., F. Tebourski, D. F. Smith, R. B. Ismail, and K. Dellagi. 1994. Antibodies to Leishmania donovani infantum heat-shock protein 70 in human visceral jeishmaniasis. J. Infect. Dis. 169:1183-1184.

31. Maalej, I. A., M. Chenick; H. Louzir; A. Ben Salah, C. Bahloul, F. Amri, and K. Dellagi. 2003. Comparative evaluation of ELISAs based on ten recombinant or purified Leishmania antigens for the serodiagnosis of Mediterranean visceral leishmaniasis. Am. J. Trop. Med. Hyg. 68:312-320.

32. Margutti P, E. Ortona, S. Vaccari, S. Barca, R. Rigano, A. Teggi, F. Muhschlegel, M. Frosch, and A. Siracusano. 1999. Cloning and expression of a cDNA encoding an elongation factor 1beta/delta protein from Echinococcus granulosus with immunogenic activity. Parasite Immunol. 21:485-492.

33. Martin S. K., L., Thuita-Harun, M., Adoyo-Adoyo and K. M., Wasunna. 1998. A diagnostic ELISA for visceral leishmaniasis, based on antigen from media conditioned by Leishmania donovani promastigotes. Ann. Trop. Med. Parasitol. 62:571-577.

34. Matsudaira, P. 1987. Sequence from picomole quantities of proteins electroblotted onto polyvinylidene difluoride membranes. J. Biol. Chem. 262:10035-10038.

35. Merrifield, R. B. 1963. Solid phase peptide synthesis. I. The synthesis of a tetrapeptide. J. Am. Chem. Soc. 85:2149-2154.

36. Mougneau, E., F. Altare, A. E. Wakil, S. Zheng, T. Coppola, Z. E. Wang, R. Waldmann, R. M. Locksley, and N. Glaichenhaus. 1995. Expression cloning of a protective Leishmania antigen. Science. 268:563-566.

37. Murray P. J, and T. W. Spithill, 1991. Variants of a Leishmania surface antigen derived from a multigenic family. J. Biol. Chem. 266:24477-24484.

38. Nandan, D., T. Yi, M. Lopez, C. Lai, and N. E. Reiner. 2002. Leishmania EF-1 alpha activates the Src homology 2 domain containing tyrosine phosphatase SHP-1 leading to macrophage deactivation. J. Biol. Chem. 277:50190-50197.

39. O'Farrell, P. Z., H. M.Goodman, and P. H. O'Farrell. 1977. “High resolution two-dimensional electrophoresis of basic as well as acid proteins. Cell. 12:1133-1142.

40. Okong'o-Odera, E. A.; A. Jorgen, L. Kurtzhals, A. S. Hey, and A. kharazmi. 1993a. Measurement of serum antibodies against native Leishmania gp63 distinguishes between ongoing and previous L. donovani infection. APMIS. 101:642-646.

41. Okong'o-Odera, E. A., A. Wamachi , J. M. Kagai, J. A. Kurtzhals, J. I. Githure, A. S. Hey., J. B. Were, D., K. Koech, E. S. Mitena, and A. Kharazmi. 1993b. Field application of an ELISA using redefined Leihmania antigen for the detection of visceral leishmaniasis. Trans. R. Soc. Trop. Med. Hyg. 87:423-424.

42. Ortona E, P. Margutti, S. Vaccari, R. Rigano, E. Profumo, B. Buttari, A. Chersi, A. Teggi, and A. Siracusano. 2001. Elongation factor 1 beta/delta of Echinococcus granulosus and allergic manifectations in human cystic echinococcosis. Clin. Exp. Immunol. 125:110-116.

43. Ouelhazi, L., M. Filali, A. Décendit, J. C. Chenieux, and M. Rideau. 1993. Differential protein accumulation in zeatin-and 2,4-D-treated cells of Catharantus roseus. Correlation with indole alkaloid biosynthesis. Plant Physiol. Biochem. 31:421-431.

44. Pearson, R. D., and A. de Queiroz Souza. 1996. Clinical Spectrum of leishmaniasis. Clin. Infect. Dis. 22:1-13.

45. Pfaff, E., M. Mussgay, H. O. Bbhm, G. E. Schulz, and H. Schaller. 1982. Antibodies against a preselected peptide recognize and neutralize foot and mouth disease virus. EMBO J. 1:869-874.

46. Priest, J. W., and S. L. Hajduk. 1996. In vitro import of the The Rieske iron-sulphur proteins by trypanosome mitochondria. J. Biol. Chem. 271:20060-20069.

47. Probst P, E. Stromberg, H. W. Ghalib, M. Mozel, R. Badaro, S. G. Reed, and J. R. Webb. 2001. Identification and characterization of T cell-stimulating antigens from Leishmania by CD4 T cell expression cloning. J. Immunol. 166:498-505.

48. Quijada, L., J. M. Requena, M. Soto, and C. Alonso. 1998. Analysis of the antigenic properties of the L. infantum Hsp70: design of synthetic peptides for specific serodiagnosis of human leishmaniasis. Immunol. Lett. 63:169-174.

49. Rabilloud, T., J. M. Strub, S. Luche, A. Van Dorsselaer, and J. Lunardi. 2001. A comparison between Sypro Ruby and ruthenium 11 tris (bathophenanthroline disulfonate) as fluorescent strains for protein detection in gels. Proteomics. 5:699-704.

50. Ramagli, L. S., and L. U. Rodriguez. 1985. Quantification of microgram amounts of proteins in Two-dimensional polyacrylamide gel electrophoresis sample buffer. Electrophoresis. 6:559-563.

51. Reed, S. G., R. Badaro, and R. M. Lioyd. 1987 Identification of specific and cross-reactive antigens of Leishmania donovani chagasi by human infection sera. J. Immunol. 138:1596-1601.

52. Requena J. M., C. Alonso, and M. Soto. 2000. Evolutionarily conserved proteins as prominent immunogens during Leishmania infections. Parasitol Today. 16:246-250.

53. Rolland L., V. Zilberfarb, A. Furtado, and M. Gentilini. 1994. Identification of a 94-kilodalton antigen on Leishmania promastigote forms and its specific recognition in human and canine visceral leishmaniasis. Parasite Immunol. 16:599-608.

54. Rolland-Burger, L., X. Rolland, C. W. Grieve, and L. Monjour. 1991. Immunoblot analysis of the humoral immune response to Leishmania donovani infantum polypeptides in human visceral leishmaniasis. J. Clin. Microbiol. 29:1429-1435.

55. Samaras, N., and T. W. Spithill. 1989. The developmentally regulated P100/11E gene of Leishmania major shows homology to a superfamily of reductase genes. J. Biol. Chem. 264:4251-4254.

56. Shreffler, W. J., J. M. Jr Burns, R. Badaro, H. W. Ghalib, L. L. Button, W. R. McMaster, and S. G. Reed. 1993. Antibody responses of visceral leishmaniasis patients to gp63, a major surface glycoprotein of Leishmania species. J. Infect. Dis. 167:426-430.

57. Soto M, J. M., Requena, L. C Gomez, I. Navarete, and C. Alonso, 1992. Molecular characterization of a Leishmania donovani infantum antigen identified as histone H2A. Eur. J. Biochem. 205:211-216.

58. Soto M, J. M. Requena, L. Quijada, M. Garcia, F. Guzman, M. E. Patarroyo, and C. Alonso. 1995. Mapping of the linear antigenic determinants from the Leishmania infantum histone H2A recognized by sera from dogs with leishmaniasis. Immunol. Lett. 48:209-214.

59. Soto M., Requena J. M., Quijada L., Perez M. J., Nieto C. G., Guzman F., Patarroyo M. E. , Alonso C. 1999. Antigenicity of the Leishmania infantum histones H2B and H4 during canine viscerocutaneous leishmaniasis. Clin. Exp. Immunol. 115:342-349.

60. Stuart M. K. 1998. An antibody diagnostic for hymenopteran parasitism is specific for a homologue of elongation factor-1 alpha. Arch. Insect. Biochem. Physiol. 39:1-8.

61. Tebourski, F., A. El Gaied, H. Louzir, R. Ben Ismail, R. Kammoun, and K. Dellagi. 1994. Identification of an immunodominant 32-kilodalton membrane protein of Leishmania donovani infantum promastigotes suitable for specific diagnosis of Mediterranean visceral leishmaniasis. J. Clin. Microbiol. 32:2474-2480.

62. Voller, A., D. E. Bidwell, and A. Bartlett. 1976. Enzyme immunoassays in diagnostic medicine. Theory and practice. Bull. W. H. O. 53:55-65.

63. Yang, F. M. Demma, M. Warren, S. Dharmawardhane, and J. Condeelis. 1990. Identification of an actin-binding protein from Dictyostelium as elongation factor 1a. Nature. 347:494-496.

What is claimed is:

1. A purified Leishmania infantum polypeptide comprising at least 10 consecutive amino acids of a protein, wherein said protein is mitochondrial integral ADP/ATP carrier protein, NADH-cytochrome b5 reductase, mitochondrial carrier protein, guanine nucleotide binding protein beta subunit (LACK), aldehyde reductase, ubiquinol-cytochrome-c reductase Rieske iron-sulfur protein, truncated elongation factor 1-alpha having a molecular weight as determined by SDS-PAGE of 36.4 kDa, truncated elongation factor 1-alpha having a molecular weight as determined by SDS-PAGE of 34.5 kDa, or truncated elongation factor 1-alpha having a molecular weight as determined by SDS-PAGE of 30.6 kDa.

2. The polypeptide of claim 1, wherein said polypeptide comprises at least 20 consecutive amino acids of said protein.

3. The polypeptide of claim 2, wherein said polypeptide comprises at least 30 consecutive amino acids of said protein.

4. The polypeptide of claim 1, wherein said polypeptide comprises a Leishmania immunodominant antigen.

5. The polypeptide of claim 1, wherein said polypeptide is recombinant.

6. A purified antibody that binds to at least one polypeptide of claim 1.

7. The purified antibody of claim 6, wherein the antibody is a monoclonal antibody.

8. A method for diagnosing Mediterranean visceral leishmaniasis (MVL), wherein the method comprises providing a composition comprising biological material suspected of being infected with Leishmania infantum, and assaying for the presence of antigens in the biological material that are immunologically reactive with an antibody of claim 6.

9. The polypeptide of claim 1, wherein said protein is mitochondrial integral ADP/ATP carrier protein.

10. The polypeptide of claim 1, wherein said protein is NADH-cytochrome b5 reductase.

11. The polypeptide of claim 1, wherein said protein is mitochondrial carrier protein.

12. The polypeptide of claim 1, wherein said protein is guanine nucleotide binding protein beta subunit (LACK).

13. The polypeptide of claim 1, wherein said protein is aldehyde reductase.

14. The polypeptide of claim 1, wherein said protein is ubiquinol-cytochrome-c reductase Rieske iron-sulfur protein.

15. The polypeptide of claim 1, wherein said protein is truncated elongation factor 1-alpha having a molecular weight as determined by SDS-PAGE of 36.4.

16. The polypeptide of claim 1, wherein said protein is truncated elongation factor 1-alpha having a molecular weight as determined by SDS-PAGE of 34.5.

17. The polypeptide of claim 1, wherein said protein is truncated elongation factor 1-alpha having a molecular weight as determined by SDS-PAGE of 30.6 kDa.

18. A diagnostic kit for detecting the presence or absence of antibodies which bind to Leishmania comprising at least one polypeptide of claim 1, and means for detecting the formation of an immune complex between said polypeptide and antibodies, wherein said means are present in an amount sufficient to perform said detection.

19. The kit of claim 18, wherein said means for detecting the formation of immune complex between the polypeptide and antibodies comprises an indirect immunofluorescence assay, a direct agglutination test, or an enzyme-linked immunosorbent assay (ELISA).

20. An immunogenic composition comprising at least one polypeptide of claim 1 in an amount sufficient to induce an immunogenic or protective response in vivo, and a pharmaceutically acceptable carrier therefor.

21. The immunogenic composition of claim 20, wherein said composition comprises a neutralizing amount of said polypeptide.

22. An immunological complex comprising a polypeptide of claim 1, and an antibody that specifically recognizes said polypeptide.

23. A method for diagnosing Mediterranean visceral leishmaniasis (MVL), wherein the method comprises providing a composition comprising biological material suspected of being infected with Leishmania infantum, and assaying for the presence of antibodies in the biological material that are immunologically reactive with a polypeptide of claim 1.

24. An in vitro diagnostic method for the detection of the presence or absence of antibodies, which bind to a polypeptide of claim 1, wherein the method comprises contacting the polypeptide with a biological fluid for a time and under conditions sufficient for the polypeptide and antibodies in the biological fluid to form an antigen-antibody complex, and detecting the formation of the complex.

25. The method of claim 24, which further comprises measuring the formation of the antigen-antibody complex.

26. The method of claim 24, wherein the formation of antigen-antibody complex is detected by immunoassay based on Western blot technique, ELISA, indirect immunofluoresence assay, or immunoprecipitation assay.

27. A mixture of purified Leishmania infantum polypeptides comprising at least two of mitochondrial integral ADP/ATP carrier protein, NADH-cytochrome b5 reductase, mitochondrial carrier protein, guanine nucleotide binding protein beta subunit (LACK), aldehyde reductase, ubiquinol-cytochrome-c reductase Rieske iron-sulfur protein, truncated elongation factor 1-alpha having a molecular weight as determined by SDS-PAGE of 36.4 kDa, truncated elongation factor 1-alpha having a molecular weight as determined by SDS-PAGE of 34.5 kDa, or truncated elongation factor 1-alpha having a molecular weight as determined by SDS-PAGE of 30.6 kDa,
wherein said mixture is essentially free of lipids.

28. The mixture of claim 27, wherein each of said polypeptides has a molecular weight of approximately 30-36 kDa as determined by SDS-PAGE.

29. The mixture of claim 27, wherein said polypeptides comprise Leishmania immunodominant antigens.

30. The mixture of claim 27, wherein said polypeptides are recombinant polypeptides.

31. A diagnostic kit for Mediterranean visceral leishmaniasis (MVL), wherein said kit comprises the mixture of claim 27, and means for detecting the formation of immune complex between antigen and antibodies, wherein the means are present in an amount sufficient to perform said detection.

32. The kit of claim 31, wherein said means for detecting the formation of immune complex between the antigen and antibodies comprises an indirect immunofluorescence assay, a direct agglutination test, or an enzyme-linked immunosorbent assay (ELISA).

33. A method for identifying a polypepitde from Leishmania promastigotes comprising the steps of:
a) providing Leishmania membrane antigens;
b) electrophoresing said membrane antigens;
c) transferring said membrane antigens to a suitable surface;
e) contacting said membrane antigens with Mediterranean visceral leishmaniasis (MVL) sera and Zoonotic cutaneous leishmaniasis (ZVL) sera under conditions sufficient to form antigen-antibody complexes;
f) detecting the formation of said antigen-antibody complexes; and
g) identifying a polypeptide that reacts with MVL sera but not ZCL sera.

34. The method of claim 33, wherein said identifying a polypeptide that reacts with MVL sera but not ZCL sera comprises amino acid sequencing.

35. The method of claim 33, wherein said identifying a polypeptide that reacts with MVL sera but not ZCL sera comprises liquid chromatography mass spectrometry.

36. The method of claim 33, wherein said polypeptide forms a complex with antibodies in the biological fluid from Mediterranean visceral leishmaniasis patients, and wherein said polypeptide does not form a complex with antibodies in the biological fluid from patients infected with Trypanosoma cruzi, Mycobacteria, malaria parasites, or amoeba.

37. The method of claim 33, wherein said method further comprises detecting a polypeptide-antibody complex by immunoassay based on Western blot technique, ELISA, indirect immunofluorescence assay, or immunoprecipitation assay.

38. The method of claim 33, wherein said electrophoresis comprises two dimensional non equilibrium pH gradient electrophoresis.

39. A purified nucleic acid encoding a polypeptide comprising at least 10 consecutive amino acids of a protein, wherein said protein is mitochondrial integral ADP/ATP carrier protein, NADH-cytochrome b5 reductase, mitochondrial carrier protein, guanine nucleotide binding protein beta subunit (LACK), aldehyde reductase, ubiquinol-cytochrome-c reductase Rieske iron-sulfur protein, truncated elongation factor 1-alpha having a molecular weight as determined by SDS-PAGE of 36.4 kDa, truncated elongation factor 1-alpha having a molecular weight as determined by SDS-PAGE of 34.5 kDa, or truncated elongation factor 1-alpha having a molecular weight as determined by SDS-PAGE of 30.6 kDa.

40. A purified nucleic acid molecule that hybridizes to either strand of a denatured, double-stranded DNA comprising the nucleic acid sequence of claim 39 under conditions of moderate stringency in 50% formamide and 6×SSC at 42° C., with washing conditions of 0.5×SSC and 0.1% SDS at 60° C.

41. A purified nucleic acid molecule that hybridizes to either strand of a denatured, double-stranded DNA comprising the nucleic acid sequence of claim 39 under conditions of high stringency in 50% formamide and 6×SSC at 42° C., with washing conditions of 0.2×SSC and 0.1% SDS at 68° C.

42. A purified nucleic acid molecule, which encodes mitochondrial integral ADP/ATP carrier protein, NADH-cytochrome b5 reductase, mitochondrial carrier protein, guanine nucleotide binding protein beta subunit (LACK), aldehyde reductase, ubiquinol-cytochrome-c reductase Rieske iron-sulfur protein, truncated elongation factor 1-alpha having a molecular weight as determined by SDS-PAGE of 36.4 kDa, truncated elongation factor 1-alpha having a molecular weight as determined by SDS-PAGE of 34.5 kDa, or truncated elongation factor 1-alpha having a molecular weight as determined by SDS-PAGE of 30.6 kDa.

43. A recombinant vector that directs the expression of a nucleic acid molecule selected from the group consisting of the purified nucleic acid molecules of any one of claims 39, 40, 41, and 42.
